(19) United States
(12) Patent Application Publication
(10) Pub. No.: US 2011/0130444 A1
(43) Pub. Date: Jun. 2, 2011

(54) METHODS AND COMPOSITIONS FOR TARGETED DELIVERY OF GENE THERAPEUTIC VECTORS

(76) Inventors: Stefan Moisyadi, Honolulu, HI (US); Ralph V. Shohet, Honolulu, HI (US); Joseph M. Kaminski, Bethesda, MD (US)

(21) Appl. No.: 12/517,520

(22) PCT Filed: May 3, 2008
(86) PCT No.: PCT/US08/05738

§ 371 Date: Dec. 10, 2010

(51) Int. Cl. A61K 31/711; A61P 43/00

(52) U.S. Cl. 514/44 R

(57) ABSTRACT

Embodiments of the present invention relate to methods and compositions for tissue- specific delivery of a gene therapeutic, transgenic nucleic acid in mammals. Methods and compositions of the invention include the steps of providing a nucleic acid comprising a transgene flanked by two terminal repeats and, within the same or on a separate nucleic acid, a nucleotide sequence encoding a transposase, wherein the transgene comprises a biotherapeutic gene, contacting the nucleic acid with perfluorocarbon gas-filled microbubbles to form a mixture, introducing the mixture into the bloodstream of a mammal, and focusing ultrasound pulses on a specific tissue of said mammal, wherein said pulses disrupt said microbubbles of said mixture and release said nucleic acid into the bloodstream within the target tissue, thereby enabling uptake of the transgenic nucleic acid into the cells of said target tissue.

### CROSS-REFERENCE TO RELATED APPLICATIONS

The present application claims the benefit of the filing date of U.S. Provisional Patent Application Ser. No. 60/927,997, filed on May 4, 2007, which is hereby incorporated by reference in its entirety.

### STATEMENT REGARDING FEDERALLY SPONSORED RESEARCH OR DEVELOPMENT

This invention was made in part with U.S. Government support under IDeA Network of Biomedical Research Excellence/National Institutes of Health Grant RR0016467-06. The U.S. Government has certain rights in the invention.

### FIELD OF THE INVENTION

The present invention relates to methods and compositions for tissue-specific delivery gene therapeutic insertional vectors for genomic integration of transgenes.

### BACKGROUND

Although gene therapy has been promoted optimistically for over a decade, the formidable technical problems and safety concerns have yet to be successfully addressed. The clinical trials to date have used inactivated viruses as vectors to shuttle transgenes into patients' cells, but these viruses are partly to blame for the devastating outcomes of such trials, which have included the development of Leukemia and even the death of some patients. The activation of oncogenes and inactivation of tumor-repressor genes has also been observed in gene therapy experiments in mice. The potential for recombination events between viral vector and endogenous retroviruses also raises the risk of generating new, more potent viruses. There have been several attempts to circumvent the problems associated with viral gene therapy, such as one approach using “gutless” viral vectors for delivery of transposons into patients' cells. However, transposons integrate into random sites in the genome, leading to insertional mutations. Other vectors undergoing testing for gene therapy utilize a bacterial site-specific recombination system called a “bacteriophage” integrase. This vector has the ability to insert large DNA fragments into cultured cells in a pseudo-site-specific manner, but is relatively ineffective in animals. The pseudo-site-specificity also introduces the risk of cancer development via the deactivation of cancer supressor genes which can contain the pseudo-sites for insertion preferred by the bacteriophage. Thus, wider application of transgenic technology will require the development of methods that provide efficient gene integration at nonrandom sites in the genome. Tissue-specific delivery of gene therapy vectors is also desirable for increased efficiency of therapeutic gene introduction in target tissues and avoidance of non-specific effects caused by introduction of the gene in other tissues.

### SUMMARY OF THE INVENTION

Embodiments of the present invention relate to methods and compositions for tissue-specific delivery of a gene therapeutic, transgenic nucleic acid in mammals. Methods and compositions of the invention include the steps of providing a nucleic acid comprising a transgene flanked by two terminal repeats and, within the same or on a separate nucleic acid, a nucleotide sequence encoding a transposase, wherein the transgene comprises a biotherapeutic gene, contacting the nucleic acid with perfluorocarbon gas-filled microbubbles to form a mixture, introducing the mixture into the bloodstream of a mammal, and focusing ultrasound pulses on a specific tissue of said mammal, wherein said pulses disrupt said microbubbles of said mixture and release said nucleic acid into the bloodstream within the target tissue, thereby enabling uptake of the transgenic nucleic acid into the cells of said target tissue.

In some embodiments of the invention, the transgene encodes a biotherapeutic polypeptide. In further embodiments of the invention, the transgene encodes a biotherapeutic ribonucleic acid product.

In some embodiments of the invention, the transposase can be selected from one of the group consisting of piggyBac, Sleeping Beauty, Mos1, Tc1/mariner, Tol2, Tc3, MuA, and Himar1 transposase.

In some embodiments of the invention, the nucleic acid comprising a transgene flanked by two terminal repeats is a piggyBac-like transposon and said transposase is a piggyBac-like transposase.

In some embodiments of the invention, the transgene is under the control of a promoter. In preferred embodiments, the transgene is under the control of the CMV promoter or CAG promoter.

In some embodiments, the transposase is a chimeric piggyBac-like transposase containing a host-specific DNA binding domain. In some embodiments, the host-specific DNA binding domain of the chimeric transposase includes a Saccharomyces cerevisiae Gal4 zinc finger DNA-binding protein. In some embodiments, the host-specific DNA binding domain of said chimeric transposase is optimized for host specificity.

In some embodiments, the transposon contains a selectable marker or reporter gene. In some embodiments, the selectable marker or reporter gene is selected from the group consisting of EGFP, luciferase, and β-galactosidase.

In some embodiments, the host-specific binding domain of the chimeric transposase is fused to the N-terminus of the transposase. In further embodiments, the host-specific binding domain is fused to the C-terminus of the transposase.

In some embodiments, the mammal is selected from one of the group consisting of primates, rodents, cows, pigs, sheep, goats, horses. In some embodiments, the mammal is a human.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 depicts a piggyBac-transposon and piggyBac transposase gene-containing plasmid encoding a luciferase reporter gene within the transposon.

FIG. 2 depicts a control plasmid with no piggyBac transposase machinery , and encoding a luciferase reporter gene.

FIG. 3 the plasmid designated pMMK-1 containing both the transposase gene and the transposon construct, including between its 5′ and 3′ terminal repeats (TRs) the gene for EGFP driven by the CAG promoter and the pSV40-hygromycin and ColE1 kanamycin resistance genes. The piggyBac transposase gene is driven by the CAG promoter.

FIG. 4 illustrates the enhanced efficiency of transgenesis in human HEK293 cells transfected with the pMMK-1 plasmid (left) relative to cells transfected with a control plasmid lacking the piggyBac transposase gene (right).

FIG. 5 depicts a new plasmid designated pMMK-2, similar to pMMK-1, but containing the piggyBac transposase gene driven by the CMV promoter and containing two kanamycin resistance genes.

FIG. 6 depicts the transposition activity of chimeric transposases containing N-terminal GAL4 DNA binding domains (A). GAL4-piggyBac retains the activity of its non-chimeric, wild type counterpart, while GAL4-SB11 and GAL4-Tol2 have negligible activity (B). illustrates the enhanced efficiency of transgenesis in human HEK293 cells transfected with the pMMK-1 plasmid (left) relative to cells transfected with a control plasmid lacking the piggyBac transposase gene (right).

FIG. 7 shows representations of two-plasmid transposon systems used to directly compare the genomic integration efficiencies of piggyBac and three other transposases. Each transposase was encoded on a helper plasmid (A), each of which was cotransfected into cultured mammalian cells along with a donor plasmid (B). The number of cell clones resistant to the antibiotic hygromycin was then measured to reveal the efficiency of genomic insertion of the pSV40-hygromycin resistance gene on the donor plasmid by each of the transposases.

FIG. 8 shows the high transposition activity of piggyBac transposase relative to three other transposases, Sleeping Beauty (SB11), Most, and Tol2, in four different mammalian cell lines, (A) HeLa, (B) H1299, (C) HEK293, and (D) CHO cells, each transfected with the plasmids from FIG. 4. (E) An example of hygromycin-resistant HEK293 cells transfected with piggyBac, Tol2, and SB11 transposon systems (from left to right), and their controls, stained for visibility. (F) PCR-based detection of transposon sequences excised from the donor plasmid in vivo by the transposase indicated, showing excision by SB11 (left) and piggyBac (middle), but not by Mos1 (right).

### ABBREVIATIONS AND DEFINITIONS

The following definitions and methods are provided to better define embodiments of the present invention and to guide those of ordinary skill in the art in the practice of embodiments of the present invention. Unless otherwise noted, terms are to be understood according to conventional usage by those of ordinary skill in the relevant art.

As used herein the term “transcription activity” refers to an activity of transcribing a DNA into an RNA (in particular, mRNA).

As used herein the term “cytomegalovirus” or “CMV” is interchangeably used to refer to a multiparticular virus, belonging to Cucumovirus group. It consists of three types of viral particles, which are all globular polyhedron, having diameter about 29 nm. The genome thereof consists of three single stranded RNAs. The virus is a plant virus whose host range is extremely broad, and is distributed all over the world as a major pathogen and viral diseases of a number of crops such as cucumber, tomatoes and the like. The promoter of Cytomegalovirus is a sequence having transcription promoting activity present in the RNA encoding the above-mentioned protein of the Cytomegalovirus. Sequences of CMV promoters are well known to those of skill in the art.

As used herein the term “CAG” promoter refers to a promoter comprising a Cytomegalovirus enhancer (e.g., Cytomegalovirus early immediate enhancer) and As used herein the term “reporter” molecule or “reporter” gene refers to a molecule (e.g. polypeptide) or gene which can be used as an indicator of gene expression in a cell. Such a molecule may be of a known reporter protein, and includes, but is not limited to for example, chloramphenicol acetyl transferase (CAT), beta-glucuronidase (GUS), beta-D-galactosidase, luciferase, green fluorescence protein (GFP), or aequorin and the like. As used herein, a method for introducing a gene per se may be achieved by means of desired material using known technology in the art. In such a case, for example, an embryonic stem cell of interest was introduced with a reporter gene free of a promoter (e.g., luciferase, green fluorescence gene, beta-galactosidase gene (lacZ), alkaline phosphatase gene, Cre recombinase gene and the like), and reporter activity will only be detected when inserted downstream of an activated promoter on the chromosome. Vectors used may include, for example, the presently mentioned reporter gene, selectable marker gene (e.g., neomycin resistant gene, hygromycin resistant gene, puromycin resistant gene, rescue marker gene (e.g., ampicillin resistant gene and collicin El replication origin) and the like. A selectable marker gene is used for selecting a host with the vector. A rescue marker gene is used for rescuing a vector (see Joyner, A. L. ed. “Gene Targeting, 2nd edition” (Oxford University Press, 2000)). Using technologies as described above, an embryonic stem cell is produced. The modified embryonic stem cell has trapped a gene. As used herein the term “trap” refers to the state where an internal gene is disrupted by insertion of a trapping vector into the genome, and the gene disrupted by the gene is marked at the same time. (chicken) beta-actin promoter related intron sequence. CAG promoter is described in, for example, Kosuga M. et al. (2000. Cell Transplant. 9(5):675-680), which is incorporated herein by reference in its entirety. Sequences of the CAG promoter are well known to those of skill in the art.

As used herein the term “cytomegalovirus enhancer” or “CMV enhancer” refers to an enhancer found in CMV, and typically includes sequences that are well known to those of skill in the art. The enhancer can be used in combination with a promoter.

As used herein, the term “cell” is herein used in its broadest sense in the art, referring to a structural unit of a tissue present in a multicellular organism, which is capable of self replicating, has genetic information and a mechanism for expressing it, and is surrounded by a membrane structure that isolates the living body from the outside. Cells used herein may be either naturally-occurring cells or artificially modified cells (e.g., fusion cells, genetically modified cells, etc), as long as the cell has a chemical receptor or is capable of having such a nucleic acid molecule introduced therein. Examples of cell sources include, but are not limited to, a single-cell culture; the embryo, blood, or a body tissue of a normally-grown transgenic animal, a mixture of cells derived from normally-grown cell lines, and the like. In some preferred embodiments, a cell which is easily transformed or transfected is used.

As used herein, the term “tissue” refers to an aggregate of cells having substantially the same function and/or form in a multi-cellular organism. “Tissue” is typically an aggregate of cells of the same origin, but may be an aggregate of cells of different origins as long as the cells have the same function and/or form. Therefore, in embodiments of the present invention, when stem cells are used to regenerate a tissue, the tissue may be composed of an aggregate of cells of two or more different origins. Typically, a tissue constitutes a part of an organ. Animal tissues are separated into epithelial tissue, connective tissue, muscular tissue, nervous tissue, and the like, on a morphological, functional, or developmental basis. Plant tissues are roughly separated into meristematic tissue and permanent tissue according to the developmental stage of the cells constituting the tissue. Alternatively, tissues may be separated into single tissues and composite tissues according to the type of cells constituting the tissue. Thus, tissues are separated into various categories.

As used herein, the term “isolated” means that naturally accompanying material is at least reduced, or preferably substantially completely eliminated, in normal circumstances. Therefore, the term “isolated cell” refers to a cell substantially free from other accompanying substances (e.g., other cells, proteins, nucleic acids, etc.) in natural circumstances. The term “isolated” in relation to nucleic acids or polypeptides means that, for example, the nucleic acids or the polypeptides are substantially free from cellular substances or culture media when they are produced by recombinant DNA techniques; or precursory chemical substances or other chemical substances when they are subsequently chemically synthesized.

As used herein, the term “gene” refers to an element defining a genetic trait. A gene is typically arranged in a given sequence on a chromosome. A gene which defines the primary structure of a protein is called a structural gene. A gene which regulates the expression of a structural gene is called a regulatory gene (e.g., promoter). As used herein, “gene” may refer to a “polynucleotide”, “oligonucleotide”, “nucleic acid”, and a “nucleic acid molecule.”

As used herein, “gene product” includes a “polynucleotide”, “oligonucleotide”, a “nucleic acid” and a “nucleic acid molecule” and/or “protein”, “polypeptide”, “oligopeptide” and a “peptide”, which are subsequent expression products of a gene. Those skilled in the art understand what a gene product is, according to the context used with embodiments of the present invention. Accordingly, gene used herein usually includes not only double-stranded DNA but also each single-stranded DNA, such as sense chain and antisense chain constituting thereof. Therefore, in embodiments of the present invention, the genes can include any of double-stranded DNA including human genome DNA, and single-stranded DNA (sense chain) including cDNA, as well as a single stranded DNA (antisense) having a sequence complementary to the sense chain, as well as fragments thereof.

The terms “polynucleotide”, “oligonucleotide”, “nucleic acid molecule” and “nucleic acid” as used herein have the same meaning and refer to a nucleotide polymer having any length. This term also includes an “oligonucleotide derivative” or a “polynucleotide derivative”. An “oligonucleotide derivative” or a “polynucleotide derivative” includes a nucleotide derivative, or refers to an oligonucleotide or a polynucleotide having linkages between nucleotides different from typical linkages, which are interchangeably used.

As used herein, the term “fragment” with respect to a polypeptide or polynucleotide refers to a polypeptide or polynucleotide having a sequence length ranging from 1 to n-1 with respect to the full length of the reference polypeptide or polynucleotide (of length n). The length of the fragment can be appropriately changed depending on the purpose. For example, in the case of polypeptides, the lower limit of the length of the fragment includes 3, 4, 5, 6, 7, 8, 9, 10, 15, 20, 25, 30, 40, 50 or more nucleotides. Lengths represented by integers which are not herein specified (e.g., 11 and the like) can be appropriate as a lower limit. For example, in the case of polynucleotides, the lower limit of the length of the fragment includes 5, 6, 7, 8, 9, 10, 15, 20, 25, 30, 40, 50, 75, 100 or more nucleotides. Lengths represented by integers which are not herein specified (e.g., 11 and the like) may be appropriate as a lower limit. As used herein, the length of polypeptides or polynucleotides can be represented by the number of amino acids or nucleic acids, respectively. However, the above-described numbers are not absolute. The above-described numbers, as the upper or lower limits, are intended to include some greater or smaller numbers (e.g., .±10%), as long as the same function is maintained. In embodiments of the present invention, it is understood that any fragment can be used as long as the fragment functions as possessing transposition activity.

### DETAILED DESCRIPTION OF THE INVENTION

Embodiments of the present invention are directed to methods of targeted delivery systems of a gene therapeutic, transgenic nucleic acid, comprising the steps of providing a nucleic acid comprising a transgene and a nucleotide sequence sequence encoding a transposase; contacting said nucleic acid with perfluorocarbon gas-filled microbubbles to form a mixture; introducing said mixture into the bloodstream of a mammal; and focusing ultrasound pulses on a specific tissue of said mammal, wherein said pulses disrupt said microbubbles of said mixture and release said nucleic acid into the bloodstream within the target tissue, thereby enabling uptake of the transgenic nucleic acid into the cells of said target tissue.

In some embodiments, the transgene is flanked by at least one terminal repeat sequence. In some embodiments, the transgene is flanked by two terminal repeat sequences.

In some embodiments, the transgene and the nucleotide sequence encoding a transposase are located on the same nucleic acid molecule. In some embodiments, the transgene and the nucleotide sequence encoding a transposase are located on separate nucleic acid molecules.

In some embodiments, the transgene comprises a biotherapeutic gene. Exemplary biotherapeutic genes include, but are not limited to, genes which regulate the cell cycle (e.g. p53, RB, mitosin); genes which induce cell death (e.g. thymidine kinase); genes which encode cytokines that augment the immunological functions of effector cells; tumor specific promoters/enhancers; tumor suppressor genes; cell cycle regulatory genes; immunomodulatory genes; cytotoxic genes; genes that induce or promote angiogenesis; reporter genes; or fragments thereof as disclosed herein.

### Microbubble Composition

The materials which can be utilized in preparing the gaseous precursor-filled lipid microspheres include any of the materials or combinations thereof known to those skilled in the art as suitable for liposome preparation. Gas precursors which undergo phase transition from a liquid to a gas at their boiling point can be used in embodiments of the present invention. The lipids used can be of either natural or synthetic origin. The particular lipids are chosen to optimize the desired properties, e.g., short plasma half-life versus long plasma half-life for maximal serum stability. It will also be understood that certain lipids can be more efficacious for particular applications, such as the containment of a therapeutic compound to be released upon rupture of the gaseous precursor-filled lipid microsphere.

The lipid in the gaseous precursor-filled liposomes can be in the form of a single bilayer or a multilamellar bilayer, and are preferably multilamellar.

Gaseous precursors which can be activated by temperature can be used in embodiments of the present invention. Table II lists examples of gaseous precursors which undergo phase transitions from liquid to gaseous states at close to normal body temperature (37° C.) and the size of the emulsified droplets that would be required to form a microsphere having a size of 10 microns. The list is composed of potential gaseous precursors that can be used to form temperature activated gaseous precursor-containing liposomes of a defined size. The list is not to be construed as being limiting by any means, as to the possibilities of gaseous precursors for the methods in embodiments of the present invention.

TABLE II
Physical Characteristics of Gaseous Precursors and Diameter
of Emulsified Droplet to Form a 10 μm Microsphere

| | | | | Diameter (μm) of |
|---|---|---|---|---|
| | | | | Emulsified droplet |
| | Molecular | Boiling Point | | to make 10 micron |
| Compound | Weight | (° C.) | Density | microsphere |
| 1- | 76.11 | 32.5 | 6.7789 | 1.2 |
| fluorobutane | | | | |
| 2-methyl | 72.15 | 27.8 | 0.6201 | 2.6 |
| butane | | | | |
| (isopentane) | | | | |
| 2-methyl 1- | 70.13 | 31.2 | 0.6504 | 2.5 |
| butene | | | | |
| 2-methyl-2- | 70.13 | 38.6 | 0.6623 | 2.5 |
| butene | | | | |
| 1-butene-3- | 66.10 | 34.0 | 0.6801 | 2.4 |
| yne-2-methyl | | | | |
| 3-methyl-1- | 68.12 | 29.5 | 0.6660 | 2.5 |
| butyne | | | | |
| perfluoro | 88.00 | −129 | 3.034 | 3.3 |
| methane | | | | |
| perfluoro | 138.01 | −79 | 1.590 | 1.0 |
| ethane | | | | |
| perfluoro | 238.03 | 3.96 | 1.6484 | 2.8 |
| butane | | | | |
| perfluoro | 288.04 | 57.73 | 1.7326 | 2.9 |
| pentane | | | | |
| octafluoro | 200.04 | −5.8 | 1.48 | 2.8 |
| cyclobutane | | | | |
| decafluoro | 238.04 | −2 | 1.517 | 3.0 |
| butane | | | | |
| hexafluoro | 138.01 | −78.1 | 1.607 | 2.7 |
| ethane | | | | |
| docecafluoro | 288.05 | 29.5 | 1.664 | 2.9 |
| pentane | | | | |
| octafluoro-2- | 200.04 | 1.2 | 1.5297 | 2.8 |
| butene | | | | |
| perfluoro | 200.04 | −5.8 | 1.48 | 2.8 |
| cyclobutane | | | | |
| octafluoro | 212.05 | 27 | 1.58 | 2.7 |
| cyclopentene | | | | |
| perfluoro | 162 | 5 | 1.602 | 2.5 |
| cyclobutene | | | | |

*Source: Chemical Rubber Company Handbook of Chemistry and Physics Robert C. Weast and David R. Lide, eds. CRC Press, Inc. Boca Raton, Florida. (1989-1990).

Examples of gaseous precursors are by no means limited to Table II. In fact, for a variety of different applications, virtually any liquid can be used to make gaseous precursors so long as it is capable of undergoing a phase transition to the gas phase upon passing through the appropriate activation temperature. Examples of gaseous precursors that can be used include, and are by no means limited to, the following: hexafluoro acetone; isopropyl acetylene; allene; tetrafluoroallene; boron trifluoride; 1,2-butadiene; 1,3-butadiene; 1,3-butadiene; 1,2,3-trichloro, 2-fluoro-1,3-butadiene; 2-methyl, 1,3butadiene; hexafluoro-1,3-butadiene; butadiyne; 1-fluoro-butane; 2-methyl-butane; decafluoro butane; 1-butene; 2-butene; 2-methy-1-butene; 3-methyl-1-butene; perfluoro-1-butene; perfluoro-1-butene; perfluoro-2-butene; 1,4-phenyl-3-butene-2-one; 2-methyl-1-butene-3-yne; butyl nitrate; 1-butyne; 2-butyne; 2-chloro-1,1,1,4,4,4-hexafluoro-butyne; 3-methyl-1-butyne; perfluoro-2-butyne; 2-bromo-butyraldehyde; carbonyl sulfide; crotononitrile; cyclobutane; methyl-cyclobutane; octafluoro-cyclobutane; perfluoro-cyclobutene; 3-chloro-cyclopentene; perfluoro ethane; perfluoro propane; perfluoro butane; perfluoro pentane; perfluoro hexane; cyclopropane; 1,2-dimethyl-cyclopropane; 1,1-dimethyl cyclopropane; 1,2-dimethyl cyclopropane; ethyl cyclopropane; methyl cyclopropane; diacetylene; 3-ethyl-3-methyl diaziridine; 1,1,1-trifluorodiazoethane; dimethyl amine; hexafluoro-dimethyl amine; dimethylethylamine; -bis-(Dimethyl phosphine)amine; 2,3-dimethyl-2-norbornane; perfluorodimethylamine; dimethyloxonium chloride; 1,3-dioxolane-2-one; perfluorocarbons such as and not limited to 4-methyl, 1,1,1,2-tetrafluoro ethane; 1,1,1-trifluoroethane; 1,1,2,2-tetrafluoroethane; 1,1,2-trichloro-1,2,2-trifluoroethane; 1,1dichloroethane; 1,1-dichloro-1,2,2,2-tetrafluoro ethane; 1,2-difluoro ethane; 1-chloro-1,1,2,2,2-pentafluoro ethane; 2-chloro, 1,1-difluoroethane; 1-chloro-1,1,2,2-tetrafluoro ethane; 2-chloro, 1,1-difluoroethane; chloroethane; chloropentafluoro ethane; dichlorotrifluoroethane; fluoro-ethane; hexafluoro-ethane; nitro-pentafluoro ethane; nitroso-pentafluoro ethane; perfluoro ethane; perfluoro ethylamine; ethyl vinyl ether; 1,1-dichloro ethylene; 1,1-dichloro-1,2-difluoro ethylene; 1,2-difluoro ethylene; Methane; Methane-sulfonyl chloride-trifluoro; Methanesulfonyl fluoride-trifluoro; Methane-(pentafluorothio)trifluoro; Methane-bromo difluoro nitroso; Methane-bromo fluoro; Methane-bromo chloro-fluoro; Methanebromo-trifluoro; Methane-chloro difluoro nitro; Methane-chloro dinitro; Methanechloro fluoro; Methane-chloro trifluoro; Methane-chloro-difluoro; Methane dibromo difluoro; Methane-dichloro difluoro; Methane-dichloro-fluoro; Methanedifluoro; Methane-difluoro-iodo; Methane-disilano; Methane-fluoro; Methaneiodo; Methane-iodo-trifluoro; Methane-nitro-trifluoro; Methane-nitroso-trifluoro; Methane-tetrafluoro; Methane-trichlorofluoro; Methane-trifluoro; Methanesulfenylchloride-trifluoro; 2-Methyl butane; Methyl ether; Methyl isopropyl ether; Methyl lactate; Methyl nitrite; Methyl sulfide; Methyl vinyl ether; Neon; Neopentane; Nitrogen (N₂); Nitrous oxide; 1,2,3-Nonadecane tricarboxylic acid-2-hydroxytrimethylester; 1-Nonene-3-yne; Oxygen (0₂); 1,4-Pentadiene; n-Pentane; Pentane-perfluoro; 2-Pentanone-4-amino-4-methyl; 1-Pentene; 2-Pentene [cis]; 2-Pentene(trans); 1-Pentene-3-bromo; 1-Pentene-perfluoro; Phthalic acid-tetrachloro; Piperidine-2,3,6-trimethyl; Propane, Propane-1,1,1,2,2,3-hexafluoro; Propane-1,2-epoxy; Propane-2,2 difluoro; Propane 2-amino, Propane-2-chloro; Propane-heptafluoro-1-nitro; Propane-heptafluoro-1-nitroso; Propane-perfluoro; Propene; Propyl-1,1,1,2,3,3-hexafluoro-2,3dichloro; Propylene-1-chloro; Propylenechloro-(trans); Propylene-2-chloro; Propylene-3-fluoro; Propylene-perfluoro; Propyne; Propyne-3,3,3-trifluoro; Styrene-3-fluoro; Sulfur hexafluoride; Sulfur (di)-decafluoro(S₂F₁₀); Toluene-2,4-diamino; Trifluoroacetonitrile; Trifluoromethyl peroxide; Trifluoromethyl sulfide; Tungsten hexafluoride; Vinyl acetylene; Vinyl ether; Xenon; Nitrogen; air; and other ambient gases.

Perfluorocarbons are the preferred gases in embodiments of the present invention, fluorine gas, perfluoromethane, perfluoroethane, perfluorobutane, perfluoropentane, perfluorohexane; even more preferrably perfluoroethane, perfluoropropane and perfluorobutane; most preferrably perfluoropropane and perfluorobutane as the more inert perfluorinated gases are less toxic.

Microspheres of embodiments of the present invention include and are not limited to liposomes, lipid coatings, emulsions and polymers.

Lipids which can be used to create lipid microspheres include but are not limited to: lipids such as fatty acids, lysolipids, phosphatidylcholine with both saturated and unsaturated lipids including dioleoylphosphatidylcholine; dimyristoylphosphatidyl-choline; dipentadecanoylphosphatidyl-choline, dilauroylphosphatidylcholine, dioleoylphosphatidyl-choline, dipalmitoylphosphatidylcholine; distearoyl-phosphatidylcholine; DL-α-phosphatidylcholine; phosphatidylethanolamines such as dioleoylphosphatidylethanolamine; DL-α-phosphatidylethanolamine; phosphatidyl-serine; phosphatidylglycerol; phosphatidylinositol, sphingolipids such as sphingomyelin; glycolipids such as ganglioside GM1 and GM2; glucolipids; sulfatides; glycosphingolipids; phosphatidic acid; palmitic acid; stearic acid; arachidonic acid; oleic acid; lipids bearing polymers such as polyethyleneglycol, chitin, hyaluronic acid or polyvinylpyrrolidone; lipids bearing sulfonated mono-, di-, oligo- or polysaccharides; cholesterol, cholesterol sulfate and cholesterol hemisuccinate; tocopherol hemisuccinate, lipids with ether and ester-linked fatty acids, polymerized lipids, diacetyl phosphate, stearylamine, cardiolipin, phospholipids with short chain fatty acids of 6-8 carbons in length, synthetic phospholipids with asymmetric acyl chains (e.g., with one acyl chain of 6 carbons and another acyl chain of 12 carbons), 6-(5-cholesten-3β-yloxy)-1-thio-β-D-galactopyranoside, digalactosyldiglyceride, 6-(5-cholesten-3β-yloxy)hexyl-6-amino-6-deoxy-1-thio-β-D-galact- opyranoside, 6-(5-cholesten-3β-yloxy)hexyl-6-amino-6-deoxyl-1-thio-α-D-mann- opyranoside, 12-(((7′-diethylaminocoumarin-3-yl)carbonyl)methylamino)octadecanoic acid; N-[12-(((7′-diethylaminocoumarin-3-yl)carbonyl)methyl-amino)-octade- canoyl]-2-aminopalmitic acid; cholesteryl).sub.4′-trimethyl-ammonio)butanoate; N-succinyldioleoylphosphatidylethanol-amine; 1,2-dioleoyl-sn-glycerol; 1,2-dipalmitoyl-sn-3-succinylglycerol; 1,3-dipalmitoyl-2-succinylglycerol; 1-hexadecyl-2-palmitoylglycero-phosphoethanolamine; and palmitoylhomocysteine; and/or combinations thereof. The liposomes can be formed as monolayers or bilayers and may or may not have a coating.

Lipids bearing hydrophilic polymers such as polyethyleneglycol (PEG), including and not limited to PEG 2,000 MW, 5,000 MW, and PEG 8,000 MW, can be used for improving the stability and size distribution of the gaseous precursor-containing liposomes. Various different mole ratios of PEGylated lipid, dipalmitoylphosphatidylethanolamine (DPPC) bearing PEG 5,000 MW, for example, can be used. A product which can be used for entrapping gaseous precursors contains, for example, 83 mole percent DPPC, 8 mole percent DPPE-PEG 5,000 MW and 5 mole percent dipalmitoylphosphatidic acid.

In addition, examples of compounds used to make mixed systems include, but by no means are limited to lauryltrimethylammonium bromide (dodecyl-), cetyltrimethylammonium bromide (hexadecyl-), myristyltrimethylammonium bromide (tetradecyl-), alkyldimethylbenzylammonium chloride (alkyl=C12,C14,C16), benzyldimethyldodecylammonium bromide/chloride, benzyldimethylhexadecylammonium bromide/chloride, benzyldimethyltetradecylammonium bromide/chloride, cetyldimethylethylammonium bromide/chloride, or cetylpyridinium bromide/chloride. Likewise perfluorocarbons such as pentafluoro octadecyl iodide, perfluorooctylbromide (PFOB), perfluorodecalin, perfluorododecalin, perfluorooctyliodide, perfluorotripropylamine, and perfluorotributylamine. The perfluorocarbons can be entrapped in liposomes or stabilized in emulsions as is well know in the art such as U.S. Pat. No. 4,865,836, which is incorporated herein by reference in its entirety. The above examples of lipid suspensions can also be sterilized via autoclave without appreciable change in the size of the suspensions.

If desired, either anionic or cationic lipids can be used to bind anionic or cationic pharmaceuticals. Cationic lipids can be used to bind DNA and RNA analogues with in or on the surface of the gaseous precursor-filled microsphere. A variety of lipids such as DOTMA, N-[1-(2,3-dioleoyloxy)propyl]-N,N,N-trimethylammonium chloride; DOTAP, 1,2-dioleoyloxy-3-(trimethylammonio)propane; and DOTB, 1,2-dioleoyl-3-(4′-trimethyl-ammonio)butanoyl-sn-glycerol can be used. In general the molar ratio of cationic lipid to non-cationic lipid in the liposome can be, for example, 1:1000, 1:100, preferably, between 2:1 to 1:10, more preferably in the range between 1:1 to 1:2.5 and most preferably 1:1 (ratio of mole amount cationic lipid to mole amount non-cationic lipid, e.g., DPPC). A wide variety of lipids can comprise the non-cationic lipid when cationic lipid is used to construct the microsphere. This non-cationic lipid can be dipalmitoylphosphatidylcholine, dipalmitoylphosphatidylethanolamine or dioleoylphosphatidylethanolamine. In lieu of cationic lipids as described above, lipids bearing cationic polymers such as polylysine or polyarginine can also be used to construct the microspheres and afford binding of a negatively charged therapeutic, such as genetic material, to the outside of the microspheres. Additionally, negatively charged lipids can be used, for example, to bind positively charged therapeutic compounds. Phosphatidic acid, a negatively charged lipid, can also be used to complex DNA. This is highly surprising, as the positively charged lipids were heretofore thought to be generally necessary to bind genetic materials to liposomes. Five to 10 mole percent phosphatidic acid in the liposomes can improve the stability and size distribution of the gaseous precursor-filled liposomes.

Other lipids or combinations thereof apparent to those skilled in the art which are in keeping with the spirit of the present invention are also encompassed by embodiments of the present invention. For example, carbohydrate-bearing lipids can be employed for in vivo targeting, as described in U.S. Pat. No. 4,310,505, which is incorporated herein by reference in its entirety.

Saturated and unsaturated fatty acids that can be used to generate gaseous precursor-filled microspheres preferably include, but are not limited to molecules that have between 12 carbon atoms and 22 carbon atoms in either linear or branched form. Examples of saturated fatty acids that can be used include, but are not limited to, lauric, myristic, palmitic, and stearic acids. Examples of unsaturated fatty acids that can be used include, but are not limited to, lauroleic, physeteric, myristoleic, palmitoleic, petroselinic, and oleic acids. Examples of branched fatty acids that can be used include, but are not limited to, isolauric, isomyristic, isopalmitic, and isostearic acids and isoprenoids.

Cationic polymers can be bound to the lipid layer through one or more alkyl groups or sterol groups which serve to anchor the cationic polymer into the lipid layer surrounding the gaseous precursor. Cationic polymers that can be used in this manner include, but are not limited to, polylysine and polyarginine, and their analogs such as polyhomoarginine or polyhomolysine. The positively charged groups of cationic lipids and cationic polymers, or perfluoroalkylated groups bearing cationic groups, for example, can be used to complex negatively charged molecules such as sugar phosphates on genetic material, thus binding the material to the surface of the gaseous precursor-filled lipid sphere. For example, cationic analogs of amphiphilic perfluoroalkylated bipyridines, as described by Garelli and Vierling (Garelli and Vierling. 2002. Biochim Biophys Acta 1127:41-48, which is incorporated herein by reference in its entirety) can be used. Alternatively, for example, negatively charged molecules can be bound directly to the head groups of the lipids via ester, amide, ether, disulfide or thioester linkages.

Bioactive materials, such as peptides or proteins, can be incorporated into the lipid layer provided the peptides have sufficient lipophilicity or can be derivatized with alkyl or sterol groups for attachment to the lipid layer. Negatively charged peptides can be attached, for example, using cationic lipids or polymers as described above.

One or more emulsifying or stabilizing agents can be included with the gaseous precursors to formulate the temperature activated gaseous precursor-filled microspheres. The purpose of these emulsifying/stabilizing agents is two-fold. Firstly, these agents can help to maintain the size of the gaseous precursor-filled microsphere. As noted above, the size of these microspheres can generally affect the size of the resultant gas-filled microspheres. Secondly the emulsifying and stabilizing agents can be used to coat or stabilize the microsphere which results from the precursor. Stabilization of contrast agent-containing microspheres can maximize the in vivo contrast effect. Although stabilization of the microsphere is preferred, it is not an absolute requirement. Because the gas-filled microspheres resulting from these gaseous precursors are more stable than air, they can still be designed to provide contrast enhancement; for example, they can pass through the pulmonary circulation following peripheral venous injection, even when not specifically stabilized by one or more coating or emulsifying agents. One or more coating or stabilizing agents is preferred, however, as are flexible stabilizing materials. Gas microspheres stabilized by polysaccharides, gangliosides, and polymers can be more effective than those stabilized by albumin and other proteins. Liposomes prepared using aliphatic compounds are preferred as microspheres stabilized with these compounds are much more flexible and stable to pressure changes.

Solutions of lipids or gaseous precursor-filled liposomes can be stabilized, for example, by the addition of a wide variety of viscosity modifiers, including, but not limited to carbohydrates and their phosphorylated and sulfonated derivatives; polyethers, preferably with molecular weight ranges between 400 and 8000; di- and trihydroxy alkanes and their polymers, preferably with molecular weight ranges between 800 and 8000. Glycerol, propylene glycol, polyethylene glycol, polyvinyl pyrrolidone, and polyvinyl alcohol can also be used as stabilizers in embodiments of the present invention. Particles which are porous or semi-solid such as hydroxyapatite, metal oxides and coprecipitates of gels, e.g. hyaluronic acid with calcium can be used to formulate a center or nidus to stabilize the gaseous precursors.

Emulsifying and/or solubilizing agents can also be used in conjunction with lipids or liposomes. Such agents include, but are not limited to, acacia, cholesterol, diethanolamine, glyceryl monostearate, lanolin alcohols, lecithin, mono- and di-glycerides, mono-ethanolamine, oleic acid, oleyl alcohol, poloxamer, polyoxyethylene 50 stearate, polyoxyl 35 castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan mono-laurate, sorbitan mono-oleate, sorbitan mono-palmitate, sorbitan monostearate, stearic acid, trolamine, and emulsifying wax. All lipids with perfluoro fatty acids as a component of the lipid in lieu of the saturated or unsaturated hydrocarbon fatty acids found in lipids of plant or animal origin can be used. Suspending and/or viscosity-increasing agents that can be used with lipid or liposome solutions include but are not limited to, acacia, agar, alginic acid, aluminum mono-stearate, bentonite, magma, carbomer 934P, carboxymethylcellulose, calcium and sodium and sodium 12, glycerol, carrageenan, cellulose, dextrin, gelatin, guar gum, hydroxyethyl cellulose, hydroxypropyl methylcellulose, magnesium aluminum silicate, methylcellulose, pectin, polyethylene oxide, polyvinyl alcohol, povidone, propylene glycol, alginate, silicon dioxide, sodium alginate, tragacanth, and xanthum gum. An exemplary product can incorporate lipid as a mixed solvent system in a ratio of 8:1:1 or 9:1:1 normal saline:glycerol:propylene glycol.

The gaseous precursor-filled liposomes of embodiments of the present invention are preferably comprised of an impermeable material. Impermeable material is defined a material that does not permit the passage of a substantial amount of the contents of the liposome in typical storage conditions. Substantial is defined as greater than about 50% of the contents, the contents being both the gas as well as any other component encapsulated within the interior of the liposome, such as a therapeutic. Preferably, no more than about 25% of the gas is released, more preferably, no more than about 10% of the gas is released, and most preferably, no more than about 1% of the gas is released during storage and prior to administration to a patient.

At least in part, the gas impermeability of gaseous precursor-filled liposomes has been found to be related to the gel state to liquid crystalline state phase transition temperature. It is believed that, generally, the higher gel state to liquid crystalline state phase transition temperature, the more gas impermeable the liposomes are at a given temperature. See Table I above and Derek Marsh, CRC Handbook of Lipid Bilayers (CRC Handbook of Lipid Bilayers, 1990, CRC Press:Boca Raton, Fla., p. 139, which is incorporated herein by reference in its entirety) for main chain melting transitions of saturated diacyl-sn-glycero-3-phosphocholines. However, it is noted that a lesser degree of energy can generally be used to release a therapeutic compound from gaseous precursor-filled liposomes composed of lipids with a lower gel state to liquid crystalline state phase transition temperature.

In certain preferred embodiments, the phase transition temperature of the lipid is greater than the internal body temperature of the patient to which they are administered. For example, lipids having a phase transition temperature greater than about 37° C. are preferred for administration to humans. In general, microspheres having a gel to liquid phase transition temperature greater than about 20° C. are adequate and those with a phase transition temperature greater than about 37° C. are preferred.

In preferred embodiments, the liposomes made by the methods of embodiments of the present invention are stable, stability being defined as resistance to rupture from the time of formation until the application of ultrasound. The lipids used to construct the microspheres can be chosen for stability. For example, gaseous precursor-filled liposomes composed of DSPC (distearoylphosphatidylcholine) can be more stable than gaseous precursor-filled liposomes composed of DPPC (dipalmitoylphosphatidylcholine) and that these in turn can be more stable than gaseous precursor-filled liposomes composed of egg phosphatidylcholine (EPC). Preferably, no more than about 50% of the liposomes rupture from the time of formation until the application of ultrasound, more preferably, no more than about 25% of the liposomes rupture, even more preferably, no more than about 10% of the liposomes, and most preferably, no more than about 1% of the liposomes.

In embodiments of the present invention, the gaseous precursor-filled liposomes maintained in aqueous solution can generally have a shelf life stability of greater than about three weeks, preferably a shelf life stability of greater than about four weeks, more preferably a shelf life stability of greater than about five weeks, even more preferably a shelf life stability of greater than about three months, and often a shelf life stability that is even much longer, such as over six months, twelve months, or two years.

In addition, in embodiments of the present invention, the gaseous precursor-filled liposomes can be stabilized with lipids covalently linked to polymers of polyethylene glycol, commonly referred to as PEGylated lipids. The incorporation of at least a small amount of negatively charged lipid into any liposome membrane can be beneficial in providing liposomes that do not have a propensity to rupture by aggregation. By at least a small amount, it is meant about 1 to about 10 mole percent of the total lipid. Suitable negatively charged lipids, or lipids bearing a net negative charge, can be readily apparent to those skilled in the art, and include, for example, phosphatidylserine, phosphatidylglycerol, phosphatidic acid, and fatty acids. For example, liposomes prepared from dipalmitoylphosphatidylcholine can be selected for their ability to rupture on application of resonant frequency ultrasound, radiofrequency energy, (e.g. microwave), and/or echogenicity in addition to their stability during delivery.

Further, the liposomes of the invention are preferably sufficiently stable in the vasculature such that they withstand recirculation. The gaseous precursor-filled liposomes can be coated such that uptake by the reticuloendothelial system is minimized. Exemplary coatings include, but are not limited to, gangliosides, glucuronide, galacturonate, guluronate, polyethyleneglycol, polypropylene glycol, polyvinylpyrrolidone, polyvinylalcohol, dextran, starch, phosphorylated and sulfonated mono, di-, tri-, oligo- and polysaccharides and albumin. The liposomes can also be coated for purposes such as evading recognition by the immune system.

The lipid used is also preferably flexible. Flexibility, as defined in the context of gaseous precursor-filled liposomes, is the ability of a structure to alter its shape, for example, in order to pass through an opening having a size smaller than the liposome.

Provided that the circulation half-life of the liposomes is sufficiently long, the liposomes can generally pass through the target tissue while passing through the body. Thus, by focusing the sound waves on the selected tissue to be treated, the therapeutic can be released locally in the target tissue. As a further aid to targeting, antibodies, carbohydrates, peptides, glycopeptides, glycolipids, lectins, and synthetic and natural polymers can also be incorporated into the surface of the liposomes. Other aids for targeting include polymers such as polyethyleneglycol, polyvinylpyrrolidone, and polyinylalcohol, which can be incorporated onto the surface via alkylation, acylation, sterol groups or derivatized head groups of phospholipids such as dioleoylphosphatidylethanolamine (DOPE), dipalmitoylphosphatidylethanolamine (DPPE), or distearoylphosphatidylethanolamine (DSPE). Peptides, antibodies, lectins, glycopeptides, oligonucleotides, and glycoconjugates can also be incorporated onto the surfaces of the gaseous precursor-filled lipid spheres.

In some embodiments, as an aid to the gaseous precursor instillation process as well as to maintain the stability of the gaseous precursor-filled liposomes, for example, emulsifiers can be added to the lipid. Examples of emulsifiers include, but are not limited to, glycerol, cetyl alcohol, sorbitol, polyvinyl alcohol, polypropylene glycol, propylene glycol, ethyl alcohol, sodium lauryl sulfate, Laureth 23, polysorbates (all units), all saturated and unsaturated fatty acids, triethanolamine, Tween 20, tween 40, Tween 60, tween 80, Polysorbate 20, Polysorbate 40, Polysorbate 60, and Polysorbate 80.

In embodiments of the present invention, for storage prior to use, the liposomes can be suspended in an aqueous solution, such as a saline solution (for example, a phosphate buffered saline solution), or simply water, and stored preferably at a temperature of between about 2° C. and about 10° C., preferably at about 4° C. Preferably, the water is sterile.

Typical storage conditions are, for example, a non-degassed aqueous solution of 0.9% NaCl maintained at 4° C. for 48 hours. The temperature of storage is preferably below the gel state to liquid crystalline state phase transition temperature of the material forming the liposomes.

Most preferably, the liposomes are stored in an isotonic saline solution, although, if desired, the saline solution can be hypotonic (e.g., about 0.3 to about 0.5% NaCl). The solution also can be buffered, if desired, to provide a pH range of about pH 5 to about pH 7.4. Suitable buffers include, but are not limited to, acetate, citrate, phosphate, bicarbonate, and phosphate-buffered saline, 5% dextrose, and physiological saline (normal saline).

Bacteriostatic agents can also be included with the liposomes to prevent bacterial degradation on storage. Suitable bacteriostatic agents include but are not limited to benzalkonium chloride, benzethonium chloride, benzoic acid, benzyl alcohol, butylparaben, cetylpyridinium chloride, chlorobutanol, chlorocresol, methylparaben, phenyl, potassium benzoate, potassium sorbate, sodium benzoate and sorbic acid.

By “gas-filled,” as used herein, it is meant liposomes having an interior volume that is at least about 10% gas, preferably at least about 25% gas, more preferably at least about 50% gas, even more preferably at least about 75% gas, and most preferably at least about 90% gas. It will be understood by one skilled in the art, once armed with the present disclosure, that a gaseous precursor can also be used, followed by activation to form a gas.

Various biocompatible gases can be employed in the gas-filled liposomes of embodiments of the present invention. Such gases include air, nitrogen, carbon dioxide, oxygen, argon, fluorine, xenon, neon, helium, or any and all combinations thereof. Other suitable gases can be apparent to those skilled in the art once armed with the present disclosure. In addition to the gaseous precursors disclosed herein, the precursors can be co-entrapped with other gases. For example, during the transition from the gaseous precursor to a gas in an enclosed environment containing ambient gas (as air), the two gases can mix and upon agitation and formation of microspheres, the gaseous content of the microspheres results in a mixture of two or more gases, dependent upon the densities of the gases mixed.

In embodiments of the present invention, the size of the liposomes can depend upon the intended use. With the smaller liposomes, resonant frequency ultrasound can generally be higher than for the larger liposomes. Sizing also serves to modulate resultant liposomal biodistribution and clearance. In addition to filtration, the size of the liposomes can be adjusted, if desired, by procedures known to one skilled in the art, such as extrusion, sonication, homogenization, the use of a laminar stream of a core of liquid introduced into an immiscible sheath of liquid. See, for example, U.S. Pat. No. 4,728,578; U.K. Patent Application GB 2193095 A; U.S. Pat. No. 4,728,575; U.S. Pat. No. 4,737,323; International Application PCT/US85/01161; Mayer et al. 1986. Biochimica et Biophysica Acta 858:161-168; Hope et al. 1985. Biochimica et Biophysica Acta 812:55-65; U.S. Pat. No. 4,533,254; Mayhew et al. 1987. Methods in Enzymology 149:64-77; Mayhew et al. 1984. Biochimica et Biophysica Acta 755: 169-74; Cheng et al. 1987. Investigative Radiology 22:47-55; PCT/US89/05040; U.S. Pat. No. 4,162,282; U.S. Pat. No. 4,310,505; U.S. Pat. No. 4,921,706; and Liposomes Technology, Gregoriadis, G., ed., Vol. 1, pp. 29-37, 51-67 and 79-108 (CRC Press Inc, Boca Raton, Fla., 1984), each of which is incorporated herein by reference in its entirety. Extrusion under pressure through pores of defined size is a preferred method of adjusting the size of the liposomes.

Since liposome size influences biodistribution, different size liposomes can be selected for various purposes. For example, for intravascular application, the preferred size range is a mean outside diameter between about 30 nanometers and about 10 microns, with the preferable mean outside diameter being about 5 microns.

More specifically, for intravascular application, the size of the liposomes is preferably about 10 μm or less in mean outside diameter, and preferably less than about 7 μm, and more preferably no smaller than about 5 nanometers in mean outside diameter. Preferably, the liposomes are no smaller than about 30 nanometers in mean outside diameter. In some embodiments, the liposomes are between about 3 μm and about 6 μm in mean outside diameter.

To provide therapeutic delivery to organs such as the liver and to allow differentiation of tumor from normal tissue, smaller liposomes, between about 30 nanometers and about 100 nanometers in mean outside diameter, are preferred.

For embolization of a tissue such as the kidney or the lung, the liposomes are preferably less than about 200 microns in mean outside diameter.

For intranasal, intrarectal or topical administration, the microspheres are preferably less than about 100 microns in mean outside diameter.

Large liposomes, e.g., between 1 and 10 microns in size, can generally be confined to the intravascular space until they are cleared by phagocytic elements lining the vessels, such as the macrophages and Kuppfer cells lining capillary sinusoids. For passage to the cells beyond the sinusoids, smaller liposomes, for example, less than about a micron in mean outside diameter, e.g., less than about 300 nanometers in size, can be utilized.

The route of administration of the liposomes can vary depending on the intended use. As one skilled in the art would recognize, in embodiments of the present invention, administration of therapeutic delivery systems can be carried out in various fashions, such as intravascularly, intralymphatically, parenterally, subcutaneously, intramuscularly, intranasally, intrarectally, intraperitoneally, interstitially, into the airways via nebulizer, hyperbarically, orally, topically, or intratumorly, using a variety of dosage forms. One preferred route of administration is intravascularly. For intravascular use, the therapeutic delivery system is generally injected intravenously, but can be injected intraarterially as well. The liposomes of the invention can also be injected interstitially or into any body cavity.

In embodiments of the present invention, the delivery of therapeutics from the liposomes using ultrasound is can be accomplished for tissues which have a good acoustic window for the transmission of ultrasonic energy. This is the case for most tissues in the body such as muscle, the heart, the liver and most other vital structures. In the brain, in order to direct the ultrasonic energy past the skull, a surgical window can be utilized. For body parts without an acoustic window, e.g. through bone, radiofrequency or microwave energy is preferred.

Additionally, the invention can be used in delivering therapeutics to a patient's lungs. Gaseous precursor-filled liposomes of embodiments of the present invention are lighter than, for example, conventional liquid-filled liposomes which generally deposit in the central proximal airway rather than reaching the periphery of the lungs. In embodiments of the present invention, the gaseous precursor-filled liposomes can improve delivery of a therapeutic compound to the periphery of the lungs, including the terminal airways and the alveoli. For application to the lungs, the gaseous precursor-filled liposomes can be applied through nebulization, for example. The resulting liposomes post nebulization can be about 1 to 2 microns in size and can float in the air. These size particles can be used for delivering drugs, peptides, genetic materials and other therapeutic compounds into the far reaches of the lung (i.e. terminal airways and alveoli). Because the gas filled liposomes are almost as light as air, much lighter than conventional water filled liposomes, they float longer in the air, and as such are better for delivering compounds into the distal lung. When DNA is added to these liposomes, it is readily adsorbed or bound to the liposomes. Thus, liposomes and microspheres filled by gas and gaseous precursors can hold vast potential for pulmonary drug delivery.

In applications such as the targeting of the lungs, which are lined with lipids, the therapeutic can be released upon aggregation of the gaseous precursor-filled liposome with the lipids lining the targeted tissue. Additionally, the gaseous precursor-filled liposomes can burst after administration without the use of ultrasound. Thus, ultrasound need not be applied to release the drug in the above type of administration.

Further, the gaseous precursor-filled liposomes of the invention can be used for therapeutics that can be degraded in aqueous media or upon exposure to oxygen and/or atmospheric air. For example, the liposomes can be filled with an inert gas such as nitrogen or argon, for use with labile therapeutic compounds. Additionally, the gaseous precursor-filled liposomes can be filled with an inert gas and used to encapsulate a labile therapeutic for use in a region of a patient that would normally cause the therapeutic to be exposed to atmospheric air, such as cutaneous and ophthalmic applications.

The gaseous precursor-filled liposomes can be used for transcutaneous delivery, such as a patch delivery system. The use of rupturing ultrasound can increase transdermal delivery of therapeutic compounds. Further, a mechanism can be used to monitor and modulate drug delivery. For example, diagnostic ultrasound can be used to visually monitor the bursting of the gaseous precursor-filled liposomes and modulate drug delivery and/or a hydrophone can be used to detect the sound of the bursting of the gaseous precursor-filled liposomes and modulate drug delivery.

In preferred embodiments, the gas-filled liposomes are administered in a vehicle as individual particles, as opposed to being embedded in a polymeric matrix for the purposes of controlled release.

For in vitro use, such as cell culture applications, the gaseous precursor-filled liposomes can be added to the cells in cultures and then incubated. Subsequently sonic energy, microwave, or thermal energy (e.g. simple heating) can be applied to the culture media containing the cells and liposomes.

Generally, the therapeutic delivery systems of the invention can be administered in the form of an aqueous suspension such as in water or a saline solution (e.g., phosphate buffered saline). Preferably, the water is sterile. Also, preferably the saline solution is an isotonic saline solution, although, if desired, the saline solution can be hypotonic (e.g., about 0.3 to about 0.5% NaCl). The solution can also be buffered, if desired, to provide a pH range of about pH 5 to about pH 7.4. In addition, dextrose can be preferably included in the media. Further solutions that can be used for administration of gaseous precursor-filled liposomes include, but are not limited to almond oil, corn oil, cottonseed oil, ethyl oleate, isopropyl myristate, isopropyl palmitate, mineral oil, myristyl alcohol, octyldodecanol, olive oil, peanut oil, persic oil, sesame oil, soybean oil, squalene, myristyl oleate, cetyl oleate, myristyl palmitate, as well as other saturated and unsaturated alkyl chain alcohols (C=2-22) esterified to alkyl chain fatty acids (C=2-22).

The dosage of gaseous precursor-filled microspheres to be administered and the mode of administration can vary depending upon the age, weight, and mammal to be treated, and the particular application (therapeutic/diagnostic) intended. Typically, dosage is initiated at lower levels and increased until the desired therapeutic effect is achieved.

### Ultrasonic Properties of Microbubbles and Administration of Ultrasound

For therapeutic drug delivery, the rupturing of the therapeutic containing liposomes of the invention is surprisingly easily carried out by applying ultrasound of a certain frequency to the region of the patient where therapy is desired, after the liposomes have been administered to or have otherwise reached that region. Specifically, it has been unexpectedly found that when ultrasound is applied at a frequency corresponding to the peak resonant frequency of the therapeutic containing gaseous precursor-filled liposomes, the liposomes can rupture and release their contents.

The peak resonant frequency can be determined either in vivo or in vitro, but preferably in vivo, by exposing the liposomes to ultrasound, receiving the reflected resonant frequency signals and analyzing the spectrum of signals received to determine the peak, using conventional means. The peak, as so determined, corresponds to the peak resonant frequency (or second harmonic, as it is sometimes termed).

Preferably, the liposomes of the invention have a peak resonant frequency of between about 0.5 megahertz and about 10 megahertz. Of course, the peak resonant frequency of the gaseous precursor-filled liposomes of the invention can vary depending on the outside diameter and, to some extent, the elasticity or flexibility of the liposomes, with the larger and more elastic or flexible liposomes having a lower resonant frequency than the smaller and less elastic or flexible liposomes.

The therapeutic-containing gaseous precursor-filled liposomes can also rupture when exposed to non-peak resonant frequency ultrasound in combination with a higher intensity (wattage) and duration (time). This higher energy, however, results in greatly increased heating, which can not be desirable. By adjusting the frequency of the energy to match the peak resonant frequency, the efficiency of rupture and therapeutic release is improved, appreciable tissue heating does not generally occur (frequently no increase in temperature above about 2° C.), and less overall energy is required. Thus, application of ultrasound at the peak resonant frequency, while not required, is most preferred.

For diagnostic or therapeutic ultrasound, any of the various types of diagnostic ultrasound imaging devices can be employed in the practice of the invention, the particular type or model of the device not being critical to the method of the invention. Also suitable are devices designed for administering ultrasonic hyperthermia, such devices being described in U.S. Pat. Nos. 4,620,546, 4,658,828, and 4,586,512, each of which is incorporated herein by reference in its entirety. Preferably, the device employs a resonant frequency (RF) spectral analyzer. The transducer probes can be applied externally or can be implanted. Ultrasound is generally initiated at lower intensity and duration, and then intensity, time, and/or resonant frequency increased until the liposome is visualized on ultrasound (for diagnostic ultrasound applications) or ruptures (for therapeutic ultrasound applications).

Although application of the various principles will be readily apparent to one skilled in the art, once armed with the present disclosure, by way of general guidance, for gaseous precursor-filled liposomes of about 1.5 to about 10 microns in mean outside diameter, the resonant frequency can generally be in the range of about 1 to about 10 megahertz. By adjusting the focal zone to the center of the target tissue (e.g., the tumor) the gaseous precursor-filled liposomes can be visualized under real time ultrasound as they accumulate within the target tissue. Using the 7.5 megahertz curved array transducer as an example, adjusting the power delivered to the transducer to maximum and adjusting the focal zone within the target tissue, the spatial peak temporal average (SPTA) power can then be a maximum of approximately 5.31 mW/cm² in water. This power can cause some release of therapeutic from the gaseous precursor-filled liposomes, but much greater release can be accomplished by using higher power.

By switching the transducer to the doppler mode, higher power outputs are available, up to 2.5 watts per cm² from the same transducer. With the machine operating in doppler mode, the power can be delivered to a selected focal zone within the target tissue and the gaseous precursor-filled liposomes can be made to release their therapeutics. Selecting the transducer to match the resonant frequency of the gaseous precursor-filled liposomes can make this process of therapeutic release even more efficient.

For larger diameter gaseous precursor-filled liposomes, e.g., greater than 3 microns in mean outside diameter, a lower frequency transducer can be more effective in accomplishing therapeutic release. For example, a lower frequency transducer of 3.5 megahertz (20 mm curved array model) can be selected to correspond to the resonant frequency of the gaseous precursor-filled liposomes. Using this transducer, 101.6 milliwatts per cm² can be delivered to the focal spot, and switching to doppler mode can increase the power output (SPTA) to 1.02 watts per cm².

To use the phenomenon of cavitation to release and/or activate the drugs/prodrugs within the gaseous precursor-filled liposomes, lower frequency energies can be used, as cavitation occurs more effectively at lower frequencies. Using a 0.757 megahertz transducer driven with higher voltages (as high as 300 volts) cavitation of solutions of gaseous precursor-filled liposomes can occur at thresholds of about 5.2 atmospheres.

Table III shows the ranges of energies transmitted to tissues from diagnostic ultrasound on commonly used instruments such as the Piconics Inc. (Tyngsboro, Mass.) Portascan general purpose scanner with receiver pulser 1966 Model 661; the Picker (Cleveland, Ohio) Echoview 8L Scanner including 80C System or the Medisonics (Mountain View, Calif.) Model D-9 Versatone Bidirectional Doppler. Generally, in embodiments of the present invention, these ranges of energies employed in pulse repetition can be used for diagnosis and monitoring the gas-filled liposomes but are insufficient to rupture the gas-filled liposomes.

TABLE III
Power and Intensities Produced by Diagnostic Equipment*

| Pulse | Total ultrasonic power | Average Intensity at |
|---|---|---|
| repetition rate (Hz) | output P (mW) | transducer face I_{TD} (W/m²) |
| 520 | 4.2 | 32 |
| 676 | 9.4 | 71 |
| 806 | 6.8 | 24 |
| 1000 | 14.4 | 51 |
| 1538 | 2.4 | 8.5 |

*Values obtained from Carson et al., Ultrasound in Med. & Biol. 1978, 3, 341-350, the disclosures of which are hereby incorporated herein by reference in their entirety.

Higher energy ultrasound such as commonly employed in therapeutic ultrasound equipment is preferred for activation of the therapeutic containing gaseous precursor-filled liposomes. In general, therapeutic ultrasound machines employ as much as 50% to 100% duty cycles dependent upon the area of tissue to be heated by ultrasound. Areas with larger amounts of muscle mass (i.e., backs, thighs) and highly vascularized tissues such as heart can require the larger duty cycle, e.g., 100%.

In diagnostic ultrasound, one or several pulses of sound are used and the machine pauses between pulses to receive the reflected sonic signals. The limited number of pulses used in diagnostic ultrasound limits the effective energy which is delivered to the tissue which is being imaged.

In therapeutic ultrasound, continuous wave ultrasound is used to deliver higher energy levels. In embodiments of the present invention, in using the liposomes, the sound energy can be pulsed. In some embodiments, the use of continuous wave ultrasound is preferred. If pulsing is employed, the sound can preferably be pulsed in echo train lengths of at least about 8 and preferably at least about 20 pulses at a time.

Either fixed frequency or modulated frequency ultrasound can be used. Fixed frequency is defined wherein the frequency of the sound wave is constant over time. A modulated frequency is one in which the wave frequency changes over time, for example, from high to low (PRICH) or from low to high (CHIRP). For example, a PRICH pulse with an initial frequency of 10 MHz of sonic energy is swept to 1 MHz with increasing power from 1 to 5 watts. Focused, frequency modulated, high energy ultrasound can increase the rate of local gaseous expansion within the liposomes and rupturing to provide local delivery of therapeutics.

The frequency of the sound used can vary from about 0.025 to about 100 megahertz. Frequency ranges between about 0.75 and about 3 megahertz are preferred and frequencies between about 1 and about 2 megahertz are most preferred. Commonly used therapeutic frequencies of about 0.75 to about 1.5 megahertz can be used. Commonly used diagnostic frequencies of about 3 to about 7.5 megahertz may also be used. For very small liposomes, e.g., below 0.5 micron in mean outside diameter, higher frequencies of sound may be preferred as these smaller liposomes can absorb sonic energy more effectively at higher frequencies of sound. When very high frequencies are used, e.g., over 10 megahertz, the sonic energy can generally have limited depth penetration into fluids and tissues. External application may be preferred for the skin and other superficial tissues, but for deep structures, the application of sonic energy via interstitial probes or intravascular ultrasound catheters may be preferred.

Where the gaseous precursor-filled liposomes are used for therapeutic delivery, the therapeutic compound to be delivered can be embedded within the wall of the liposome, encapsulated in the liposome and/or attached to the liposome, as desired. The phrase “attached to” or variations thereof, as used herein in connection with the location of the therapeutic compound, means that the therapeutic compound is linked in some manner to the inside and/or the outside wall of the microsphere, such as through a covalent or ionic bond or other means of chemical or electrochemical linkage or interaction. The phrase “encapsulated in variations thereof' as used in connection with the location of the therapeutic compound denotes that the therapeutic compound is located in the internal microsphere void. The phrase “embedded within” or variations thereof as used in connection with the location of the therapeutic compound, signifies the positioning of the therapeutic compound within the microsphere wall. The phrase “comprising a therapeutic” denotes all of the varying types of therapeutic positioning in connection with the microsphere. Thus, the therapeutic can be positioned variably, such as, for example, entrapped within the internal void of the gaseous precursor-filled microsphere, situated between the gaseous precursor and the internal wall of the gaseous precursor-filled microsphere, incorporated onto the external surface of the gaseous precursor-filled microsphere and/or enmeshed within the microsphere structure itself.

Therapeutics and Incorporation into Microbubbles

In embodiments of the present invention, examples of genetic therapeutics that can be applied using the liposomes include a nucleic acid comprising a transgene and a nucleotide sequence encoding a transposase. In some embodiments, the transgene and the nucleotide sequence encoding a transposase are located on the same nucleic acid molecule. In some embodiments, the, transgene and the nucleotide sequence encoding a transposase are located on separate nucleic acid molecules.

Transposons and transposases derived therefrom can be of bacterial origin. However, in a preferred embodiment of the present invention, the transposase or a fragment or derivative thereof having transposase function is a eukaryotic transposase or a fragment of or derived from a eukaryotic transposase. The transposase can be derived from a class I or class II transposon.

Exemplary transposases include, but are not limited to, at least one of the following: piggyBac, Sleeping Beauty, Mos1, Tc1/mariner, Tol1, Tc3, MuA, Himar I, Frog Prince, and derivatives thereof The piggyBac transposon and transposase are described, for example, in U.S. Patent Application Publication No. 2007/0204356, which is incorporated herein by reference in its entirety. The Sleeping Beauty transposon and transposase are described, for example, in Izsvak et al. (2000. J. Mol. Biol. 302: 93-102), which is incorporated herein by reference in its entirety. The Frog Prince transposon and transposase are described, for example, in German patent application no. DE 102 24 242.9 and in Miskey et al. (2003. Nucleic Acids Res. 31:6873-6881), each of which is incorporated herein by reference in its entirety. Other transposons and transposases are well known to those of skill in the art.

In some embodiments, the DNA level transposase enzymes can operate as a two plasmid system that includes: (1) a helper plasmid that expresses the transposase, and (2) a donor plasmid that contains the transposon. Such systems, including the transposase known as piggyBac, are described as having a helper plasmid expressing the transposase in the “trans” position to the donor plasmid (U.S. Pat. No. 6,962,810, which is incorporated herein by reference in its entirety). In other embodiments, construction of a single plasmid can be achieved by joining the helper and donor plasmids and eliminating the redundant sequences in the original helper and donor constructs. For example, a single plasmid transposase system is described in International Application No. PCT/US07/18922, filed Aug. 28, 2007, which is incorporated herein by reference in its entirety.

In addition, chimeric transposases comprising a host-specific DNA binding domain can be used. Exemplary chimeric transposases are described, for example, in U.S. Patent Application Publication No. 2006/0210977, which is incorporated herein by reference in its entirety. In a specific example, the use of a chimeric piggyBac transposase system, including the DNA binding domains of transcription factors in gene therapy proceduresis, is described in International Patent Application No. PCT/US07/18922 (supra). Such DNA binding domains recognize and bind to specific DNA sequences within or near a particular gene sequence. Some classes of transcription factors are characterized by their zinc binding capacity and are known as zinc finger DNA binding proteins (ZFPs). The DNA recognition and binding function of ZFPs can be used to target a variety of functional domains in a gene-specific location. The recognition domain of ZFPs is composed of two or more zinc fingers; each finger recognizes and binds to a three base pair sequence of DNA and multiple fingers can be linked together to more precisely recognize longer stretches of DNA. Thus, in a particular embodiment, chimeric transposases with engineered ZPFs whose DNA-interacting amino acid residues can be modified to recognize specific DNA sequences in variety of different genes can be used. Such systems are also described in International Patent Application No. PCT/US07/18922 (supra).

Exemplary transgenes can include a “therapeutic gene,” which refers to a nucleic acid sequence which encodes a protein having a therapeutically beneficial effect such as regulating the cell cycle or inducing cell death. Examples of genes which regulate the cell cycle include p53, RB and mitosin whereas a gene which induces cell death includes the conditional suicide gene thymidine kinase. Cytokines which augment the immunological functions of effector cells are also included within the term as defined herein.

Included within the definition of the therapeutic genes of the invention are active fragments thereof and genes which contain minor modifications which do not significantly effect the intended function of the gene product. Thus, “active fragments” of therapeutic genes include smaller portions of the gene that retain the ability to encode proteins having therapeutic benefit. p56^{RB}, is an example of an active fragment of a therapeutic gene which is a tumor suppressor gene. Modifications of therapeutic genes which are contemplated include nucleotide additions, deletions or substitutions, so long as the functional activity of the unmodified gene is retained. Thus, such modifications result in equivalent gene products that depart from the linear sequence of the naturally occurring proteins or polypeptides, but which have amino acid substitutions that do not change its biological activity. These equivalents can differ from the native sequences by the replacement of one or more amino acids with related amino acids, for example, similarly charged amino acids, or the substitution or modification of side chains or functional groups.

In some embodiments, the transgene can include a “tumor specific gene regulatory region” otherwise known as a “tumor specific regulatory region” or “tumor specific promoter” or “tumor specific promoter/enhancer,” all of which refer to transcription and/or translation regulatory regions that function selectively or preferentially in a specific tumor cell type. Selective or preferential function confers specificity to the gene therapy treatment since the therapeutic gene will be primarily expressed in a targeted or specific tumor cell type. Tumor specific regulatory regions include transcriptional, mRNA maturation signals and translational regulatory regions that are tumor cell type specific. Transcriptional regulatory regions include, for example, promoters, enhancers and silencers. Specific examples of such transcriptional regulatory regions include the promoter/enhancer elements for alpha-fetoprotein, carcinoembryonic antigen and prostate specific antigen. RNA processing signals include, for example, tissue specific intron splicing signals whereas translational regulatory signals can include, for example, mRNA stability signals and translation inition signals. Thus, tumor specific regulatory regions include all elements that are essential for the production of a mature gene product in a specific tumor cell type.

In some embodiments, the transgene can include a “tumor suppressor gene,” which refers to a gene that encodes a protein that effectively inhibits a cell from behaving as a tumor cell. A specific example of a tumor suppressor gene is the retinoblastoma (RB) gene. The complete RB cDNA nucleotide sequences and predicted amino acid sequences of the resulting RB protein (designated p110^{RB}R) are shown in Lee et al. (1987. Nature 329:642-645, which is incorporated herein by reference in its entirety). A truncated version of p110^{RB}R, called p56^{RB} also functions as a tumor suppressor gene and is therefore useful as a therapeutic gene. The sequence of p56^{RB} is described by Huang et al. (1991. Nature 350:160-162, which is incorporated herein by reference in its entirety). Tumor suppressor genes other than RB include, for example, the p16 protein (Kamb et al. 1994. Science 264:436-440, which is incorporated herein by reference in its entirety), p21 protein, Wilm's tumor WT1 protein, or colon carcinoma DCC protein or related molecules such as mitosin and H-NUC. Mitosin is described in U.S. Pat. No. 5,710,022, which is incorporated herein by reference in its entirety. Sequences of these exemplary tumor suppressor genes are well known to those of skill in the art.

Also encompassed within the definition of a tumor suppressor protein is any protein whose presence suppresses the neoplastic phenotype by reducing or eliminating the tumorigenicity, malignancy or hyperproliferative phenotype of the host cell. The neoplastic phenotype is characterized by altered morphology, faster growth rate, higher saturation density, growth in soft agar and tumorigenicity. The therapeutic genes described above encode proteins which exhibit this activity. “Tumorigenicity” is intended to mean having the ability to form tumors or capable of causing tumor formation and is synonymous with neoplastic growth. “Malignancy” is intended to describe a tumorigenic cell having the ability to metastasize and endanger the life of the host organism.

Tumor suppressor genes are well known to those of skill in the art and include, but are not limited to RB, p53, APC, FHIT (see, e.g., Siprashvili. 1997. Proc. Natl. Acad. Sci. USA 94:13771-13776, which is incorporated herein by reference in its entirety), BRCA1 and BRCA2, VHL, WT, DCC, FAP, NF, MEN, E-cadherin, nm23, MMACI, and PTC. The RB or retinoblastoma gene is the prototypical tumor suppressor and has been well characterized (see, e.g., Bookstein. 1990. Science 247: 712-715; Benedict. 1980. Cancer Invest. 8: 535-540; Riley. 1990. Ann. Rev. Cell Biol. 10:1-29; and Wienberg. 1992. Science 254: 1138-1146, each of which is incorporated herein by reference in its entirety). Perhaps the best characterized tumor suppressor is p53 which has been implicated in many neoblastomas as well as in the genetic predisposition to the development of diverse tumors in families with Li-Fraumeni syndrome (see, e.g., Wills. 1994. Hum. Gene Therap. 5:1079-1088; U.S. Pat. No. 5,532,220; WO 95/289048; and Harris. 1996. J. Nat. Canc. Inst. 88(20):1442, each of which is incorporated herein by reference in its entirety) which describe the cloning expression and use of p53 in gene therapy). Other tumor suppressors include WT (i.e., WT1 at 11p13) gene characteristic of Wilms' tumor (see Call et al. 1990. Cell 60:509-520; Gessler. 1990. Nature 343:774-778; and Rose et al. 1990. Cell 60:495-508, each of which is incorporated herein by reference in its entirety). The tumor suppressor gene called FHIT, for Fragile Histidine Triad, was found in a region on chromosome 3 (3p14.2, also reported at 3p21) that is known to be particularly prone to translocations, breaks, and gaps is believed to lead to esophageal, stomach and colon cancers (see, e.g., Ohta et al. 1996. Cell 84:587-597, GenBank Accession No: U469227, each of which is incorporated herein by reference in its entirety). The tumor suppressor genes DCC (18q21) and FAP are associated with colon carcinoma (see, e.g., Hedrick et al. (1994) Genes Dev., 8(10): 1174-1183; GenBank Accession No: X76132 for DCC, and Wienberg (1992) Science, 254: 1138-1146, each of which is incorporated herein by reference in its entirety). The NF tumor suppressors (NF1 at 17q11 and NF2 at 22q12) are associated with neurological tumors (e.g., neurofribromatosis for NF1, see, e.g., Caivthon et al. (1990) Cell, 62: 193-201; Viskochil et al. (1990) Cell, 62: 187-192; Wallace et al. (1990) Science, 249: 181-186; and Xug et al. (1990) Cell, 62: 599-608, each of which is incorporated herein by reference in its entirety, and Meningioma and schwannoma for NF2). The MEN tumor suppressor is associated with tumors of the multiple endocrine neoplasia syndrome (see, e.g., Wienberg, Science, 254: 1138-1146, and Marshall (1991) Cell, 64: 313-326, each of which is incorporated herein by reference in its entirety). The VHL tumor suppressor is associated with von Hippel-Landau disease (Latif (1993) Science, 260: 1317-1320, GenBank Accession No: L15409, each of which is incorporated herein by reference in its entirety). The widely publicized BRCA1 and BRCA2 genes are associated with breast cancer (see, e.g., Skolnick (1994) Science, 266: 66-71, GenBank Accession No: U14680 for BRCA1, and Teng (1996) Nature Genet., 13:241-244; GenBank Accession No: U43746, each of which is incorporated herein by reference in its entirety). In addition, the E-cadherin gene is associated with the invasive phenotype of prostate cancer (Umbas (1992) Cancer Res., 52: 5104-5109, Bussemakers (1992) Cancer Res., 52: 2916-2999, GenBank Accession No: 272397, each of which is incorporated herein by reference in its entirety). The NM23 gene is associated with tumor metastasis (Dooley (1994) Hum. Genet., 93(1): 63-66, GenBank Accession No: X75598, each of which is incorporated herein by reference in its entirety). Other tumor suppressors include DPC4 (identified at 18q21) associated with pancreatic cancer, hMLH1 (3p) and hMSH2 (2p) associated with colon cancers, and CDKN2 (16) and (9p) associated with melanoma, pancreatic and esophageal cancers. Finally, the human PTC gene (a homologue of the drosophila patched (ptc) gene) is associated with nevoid basal cell carcinoma syndrome (NBCCS) and with somatic basal cell carcinomas (see, e.g., see Hahn et al. (1996) Cell, 85: 841-851, which is incorporated herein by reference in its entirety). This list of tumor suppressor genes is neither exhaustive nor intended to be limiting and is meant simply to illustrate the wide variety of known tumor suppressors.

In some embodiments, the transgene can include a “cell cycle regulatory gene,” which refers to genes encoding proteins which directly or indirectly control one or more regulatory steps within the cell cycle. Such cell cycle regulatory steps include, for example, the control of quiescent to proliferative phenotypes such as the 0ₒ/G₁ transition as well as progression into apoptosis. Examples of cell cycle regulatory genes include the cyclins and cyclin dependent kinases.

In some embodiments, the transgene can include an “immunomodulatory gene,” which refers to genes encoding proteins which either directly or indirectly have an effect on the immune system which augments the host's inherent response toward proliferating tumor cells. Such immunomodulatory genes include, for example, cytokines such as interleukins and interferons which are recognized by effector cells of the immune system.

In some embodiments, the transgene can include a “cytotoxic gene,” which refers to a gene that encodes a protein which either alone or in combination with other agents is lethal to cell viability. Examples of cytotoxic genes which alone are lethal include toxins such as pertussis toxin, diphtheria toxin and the like. Examples of cytotoxic genes which are used in combination with other agents to achieve cell lethality include, for example, herpes simplex-1 thymidine kinase and cytosine deaminase. The subject is then administered an effective amount of a therapeutic agent, which in the presence of the anti-tumor gene is toxic to the cell. In the specific case of thymidine kinase, the therapeutic agent is a thymidine kinase substrate such as ganciclovir (GCV), 6-methoxypurine arabinonucleoside (araM), or a functional equivalent thereof Both the thymidine kinase gene and the thymidine kinase metabolite must be used concurrently to be toxic to the host cell. However, in its presence, GCV is phosphorylated and becomes a potent inhibitor of DNA synthesis whereas araM gets converted to the cytotoxic anabolite araATP. Other anti-tumor genes can be used as well in combination with the corresponding therapeutic agent to reduce the proliferation of tumor cells. Such other gene and therapeutic agent combinations are known by one skilled in the art. Another example would be the vector of this invention expressing the enzyme cytosine deaminase. Such vector would be used in conjunction with administration of the drug 5-fluorouracil, or the E. coli Deo A gene in combination with 6-methyl-purine-2′-deosribonucleoside (Sorscher et al. 1994. Gene Ther 1:233-238, which is incorporated herein by reference in its entirety).

In some embodiments, the transgene can include therapeutic genes for inducing or promoting angiogenesis. Exemplary angiogenesis therapeutic genes include, but are not limited to, sequences encoding NO synthase (“NOS”), bFGF, VEGF, EGF, tumor necrosis factor (TNF-α), interleukin-1 (IL-1), keratinocyte growth factor (KGF), interferon, or any combinations of these genes thereof VEGF includes VEGF isomers, such as VEGF-B, VEGF-C and VEGF-D. VEGF-B has been isolated and characterized (Grimmond et al., 1996, Genome Research 6: 124-131; Olofsson et al., 1996, Proc. Natl. Acad. Sci. USA 93: 2576-2581, each of which is incorporated herein by reference in its entirety). VEGF-C has also been isolated and characterized (Joukov et al., 1996, EMBO J. 15: 290-298; PCT International application WO 96/39515, each of which is incorporated herein by reference in its entirety). VEGF-D was identified in an EST library, the full-length coding region was cloned and recognized to be most homologous to VEGF-C among the VEGF family amino acid sequences (Yamada, et al., 1997, Genomics 42:483-488, which is incorporated herein by reference in its entirety).

VEGF and its homologues impart activity by binding to vascular endothelial cell plasma membrane-spanning tyrosine kinase receptors which then activate signal transduction and cellular signals. The Flt receptor family is a major tyrosine kinase receptor which binds VEGF with high affinity. At present the fit receptor family includes flt-1 (Shibuya, et al., 1990, Oncogene 5: 519-524), KDR/flk-1(Terman, et al., 1991, Oncogene 6: 1677-1683; Terman, et al., 1992, Biochem. Biophys. Res. Commun. 187: 1579-1586), and flt-4 (Pajusola, et al., 1992, Cancer Res. 52: 5738-5743, each of which is incorporated herein by reference in its entirety).

In some embodiments, the therapeutic genes are under the control of a inducible promoter so that preferential tissue specific expression relies on the tumor specific expression of an essential replication gene. In other embodiments, the therapeutic genes can similarly be under the control of a tumor specific gene regulatory region. The combined tumor specific expression of both a replication gene and the therapeutic gene is can advantageously provide greater specificity and therefore provide greater efficacy.

In some embodiments, the therapeutic genes are comprised within expression cassettes that can be incorporated into the liposomes or microbubbles of the invention to allow greater flexibility to modify the vectors with a variety of genes necessary for a particular application. An expression cassette is therefore a functional term to describe the ability of the vector to achieve the recombinant production of the therapeutic gene of interest.

In some embodiments, the genetic therapeutic can further include a reporter gene. As used herein, the term “reporter” molecule or “reporter” gene refers to a molecule (e.g. polypeptide) or gene which can be used as an indicator of gene expression in a cell or tissue. Such a molecule can be of a known reporter protein, and includes, but is not limited to for example, chloramphenicol acetyl transferase (CAT), beta-glucuronidase (GUS), beta-D-galactosidase, luciferase, green fluorescence protein (GFP), aequorin, firefly luciferase, and the like. The nucleic acid vectors used can include, for example, a reporter gene, a selectable marker gene (e.g., neomycin resistant gene, hygromycin resistant gene, puromycin resistant gene, rescue marker gene (e.g., ampicillin resistant gene and collicin El replication origin) and the like. A selectable marker gene can be used for selecting a host with the vector. A rescue marker gene can be used for rescuing a vector (see Joyner, A. L. ed. “Gene Targeting, 2nd edition” (Oxford University Press, 2000)). Specific exemplary vectors include, but are not limited to, those derived from pGL3 vector (Promega).

The genetic materials as disclosed herein can be incorporated into the internal gaseous precursor-filled space of these liposomes during the gaseous precursor installation process or into or onto the lipid membranes of these particles. Incorporation onto the surface of these particles is preferred. Genetic materials with a high octanol/water partition coefficient can be incorporated directly into the lipid layer surrounding the gaseous precursor but incorporation onto the surface of the gaseous precursor-filled lipid spheres can also be accomplished. To accomplish this, groups capable of binding genetic materials or bioactive materials are generally incorporated into the lipid layers which can then bind these materials. In the case of genetic materials, this is readily accomplished through the use of cationic lipids or cationic polymers which can be incorporated into the dried lipid starting materials.

Preferred genetic materials include such as nucleic acids, RNA, and DNA, of either natural or synthetic origin, including recombinant RNA and DNA and antisense RNA and DNA. Types of genetic material that can be used include, for example, genes carried on expression vectors such as plasmids, phagemids, cosmids, yeast artificial chromosomes (YACs), and defective or “helper” viruses, antigene nucleic acids, both single and double stranded RNA and DNA and analogs thereof, such as phosphorothioate, phosphoroamidate, and phosphorodithioate oligodeoxynucleotides. Additionally, the genetic material can be combined, for example, with proteins or other polymers.

In some embodiments, the genetic material includes DNA or RNA sequences that block gene expression. Exemplary sequences include siRNA or RNAi. In such embodiments, the phrase “at least a portion of means that the entire gene need not be represented by the nucleic acid molecule, so long as the portion of the gene represented provides an effective block to gene expression.

The liposomes can also be designed so that there is a symmetric or an asymmetric distribution of the drug both inside and outside of the liposome.

The particular chemical structure of the therapeutic gene can be selected or modified to achieve desired solubility such that the therapeutic can either be encapsulated within the internal gaseous precursor-filled space of the liposome, attached to the liposome or enmeshed in the liposome. The surface-bound therapeutic can bear one or more acyl chains such that, when the bubble is popped or heated or ruptured via cavitation, the acylated therapeutic can then leave the surface and/or the therapeutic can be cleaved from the acyl chains chemical group. Similarly, other therapeutics can be formulated with a hydrophobic group which is aromatic or sterol in structure to incorporate into the surface of the liposome.

In some embodiments, the methods provide for shaking an aqueous solution comprising a lipid and therapeutic in the presence of a temperature activated gaseous precursor, wherein the therapeutic comprises a nucleic acid molecule comprises a transgene and a nucleotide sequence encoding a transposase. In some embodiments, the transgene and a nucleotide sequence encoding a transposase are provided on separate nucleic acid molecules. Shaking, as used herein, is defined as a motion that agitates an aqueous solution such that gaseous precursor is introduced from the local ambient environment into the aqueous solution. Any type of motion that agitates the aqueous solution and results in the incorporation of the therapeutic into the liposome. Preferably, the shaking is of sufficient force such that incorporation of the therapeutic into the liposome is achieved, such as 30 minutes, and preferably within 20 minutes, and more preferably, within 10 minutes. The shaking can be by microemulsifying, by microfluidizing, for example, swirling (such as by vortexing), side-to-side, or up and down motion. In the case of the addition of gaseous precursor in the liquid state, sonication can be used in addition to the shaking methods set forth above. Further, different types of motion can be combined. Also, the shaking can occur by shaking the container holding the aqueous solution, or by shaking the aqueous solution within the container without shaking the container itself. Further, the shaking can occur manually or by machine. Mechanical shakers that can be used include, for example, a shaker table, such as a VWR Scientific (Cerritos, Calif.) shaker table, a microfluidizer, Wig-L-Bug.™ (Crescent Dental Manufacturing, Inc., Lyons, Ill.) and a mechanical paint mixer, as well as other known machines. Another means for producing shaking includes the action of gaseous precursor emitted under high velocity or pressure. It will also be understood that preferably, with a larger volume of aqueous solution, the total amount of force will be correspondingly increased. Vigorous shaking is defined as at least about 60 shaking motions per minute, and is preferred. Vortexing at at least 1000 revolutions per minute, an example of vigorous shaking, is more preferred.

### Targets

In embodiments of the invention, the methods and compositions disclosed herein can be applied to any target organ or target tissue.

As used herein, the term “tissue” refers to an aggregate of cells having substantially the same function and/or form in a multi-cellular organism. “Tissue” is typically an aggregate of cells of the same origin, but may be an aggregate of cells of different origins as long as the cells have the same function and/or form. Therefore, if stem cells are used to regenerate a tissue, the tissue may be composed of an aggregate of cells of two or more different origins. Typically, a tissue constitutes a part of an organ. Animal tissues are separated into epithelial tissue, connective tissue, muscular tissue, nervous tissue, and the like, on a morphological, functional, or developmental basis. Alternatively, tissues may be separated into single tissues and composite tissues according to the type of cells constituting the tissue. Thus, tissues are separated into various categories.

In embodiments of the present invention, any organ may be targeted. Furthermore, in embodiments of the present invention, a target tissue or cell can also be derived from any organ. As used herein, the term “organ” refers to a morphologically independent structure localized at a particular portion of an individual organism in which a certain function is performed. In multi-cellular organisms (e.g., animals, plants), an organ consists of several tissues spatially arranged in a particular manner, each tissue being composed of a number of different cells. An example of such an organ includes an organ relating to the vascular system. In one embodiment, organs targeted include, but are not limited to, skin, blood vessel, cornea, kidney, heart, liver, umbilical cord, intestine, nerve, lung, placenta, pancreas, brain, peripheral limbs, retina, and the like.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate embodiments of the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

Example 1

### Microbubble Delivery of a PiggyBac Insertional Plasmid to Heart Tissue in Mice

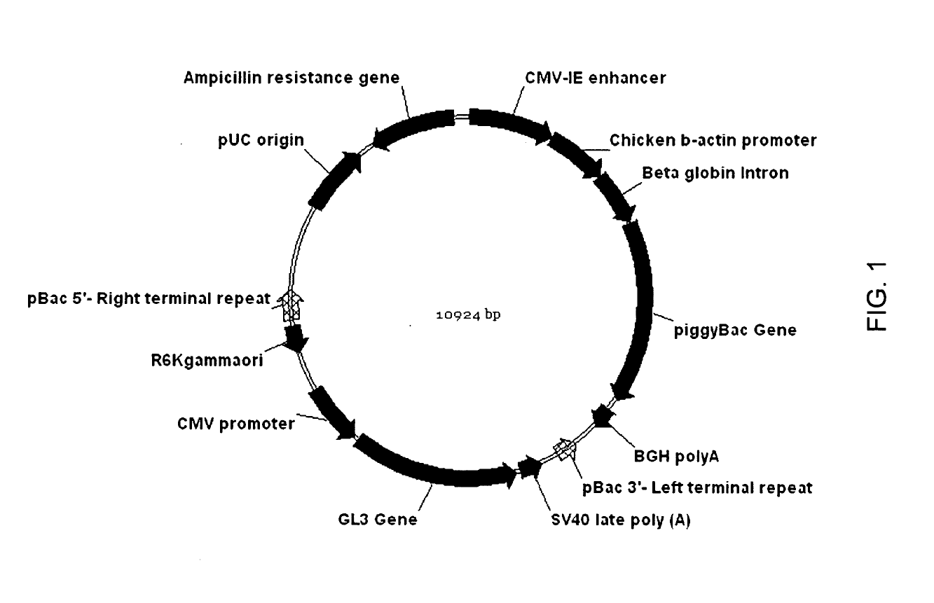

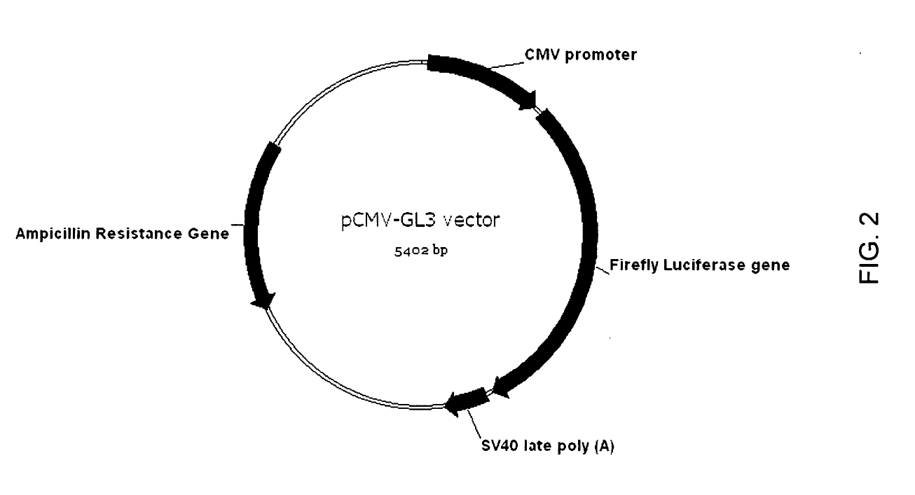

A piggyBac insertional vector was specifically deliverd to heart tissue in mice using the following procedure. A plasmid carrying 1) the gene for piggyBac transposase and 2) a piggyBac transposon containing the gene for firefly luciferase under the control of the CMV promoter was first constructed. This plasmid, denoted DH2pGL3 (FIG. 1) contains the firefly luciferase (GL3) gene and promoter between the two terminal repeats recognized by the piggyBac transposase enzyme, which excises the intervening region at these sites and inserts the segment into genomic DNA at TTAA sites. To control for the level of luciferase expressed by transfected but non-integrated GL3 gene, the plasmid pCMV-GL3 (FIG. 2), which contains GL3 under the control of the CMV promoter, but lacks the piggyBac transposase gene and the piggyBac terminal repeat sequences, was tested in parallel with DH2pGL3. To incorporate test plasmid into the shell of perfluorocarbon gas-filled cationic microbubbles, 0.5 ml of a solution containing 2 mg plasmid, 1% DL-α-phosphatidylcholine, 0.25% DL-α-phosphtidylethanolamine and 10% glycerol in PBS was incubated at 40° C. for 30 minutes. The solution was then transferred to a tube containing 1 ml of perfluorcarbon gas and shaken vigorously for 20 seconds to produce microbubbles of 3-6 μm diameter. The amount of bound plasmid in each resulting solution is approximately 200 μg. The resulting microbubbles were washed three times with Phosphate-buffered saline (PBS) by floating the microbubbles to the top of the suspension using brief centrifugation at 400×g in a swinging-bucket rotor followed by exchange of the infranatant buffer with fresh PBS. These washes ensure that only bound plasmids are infused into the animals. After 3 washes, the microbubbles were diluted with PBS to one ml prior to use. The plasmid-microbubble solution is then infused intravenously into anesthetized mice through the tail vein at a concentration of 400±20 μg/ml for 20 minutes at a constant rate of 3 ml/h. Throughout the infusion, microbubble destruction was achieved by directing ultrasound at the heart using 1 MHz pulsing with a mechanical index of 1.3, while target tissue and bubble perfusion were concurrently visualized with a 30 MHz transducer. The ultrasound pulses were ECG-triggered (at 80 ms after the peak of the R wave) to deliver a burst of four frames of ultrasound every four cardiac cycles. At the end of each experiment, all mice were sutured and monitored for normal behavior. Mice were killed either 2 or 28 days later, and hearts were removed to assay levels of luciferase expression. To test for organ-specificity of luciferase gene delivery, hearts were removed from mice given liver UTMD treatment, and assayed for luciferase expression in parallel.

To quantitate expression of the luciferase transgene in the UTMD-targetted hearts, each isolated heart was homogenized in a glass dounce in luciferase lysis buffer (Promega)/0.1% Nonidet P-40/0.5% deoxycholate/proteinase inhibitors. The resulting homogenate was centrifuged at 10,000×g for 10 minutes, and 100 μl of luciferase reaction buffer (Promega) was added to 20 μl of the clarified supernatant. Light emission was measured by a GloMax™ luminometer (Promega) in Relative Luminescence Units (RLU). The total protein content of test samples was determined using the Bradford assay (Bio-Rad protein assay), and luciferase activity was standardized to equal amounts of protein for all samples. A solution of purified, recombinant GL3 luciferase was used as a positive control, and assay buffer was used as negative control.

The specificity of tissue delivery was evident by the absence of luciferase expression in hearts isolated from mice given liver UTMD. In mice infused with the piggyBac insertional plasmid DH2pGL3, luciferase expression was observed in the heart at both 2 and 28 days after UTMD. At two days, the expression was significantly greater than the luciferase expression in mice infused with SV40pGL3-loaded microbubbles (which indicated the background levels of luciferase expressed from transfected luciferase gene that cannot be inserted into genomic DNA by piggyBac transposase, which is not encoded on the SV40pGL3 control plasmid). In hearts isolated from mice 28 days following infusion with the piggyBac plasmid DH2pGL3 and heart UTMD, luciferase expression was visible at a level significantly higher than background, indicating that the luciferase gene was maintained in the cells following genomic insertion by piggyBac. No luciferase expression was observed in SV40pGL3-UTMD-treated mice at 28 days. Thus the luciferase gene-containing piggyBac transposon was integrated into the genome of heart cells, as expression would not be visible from an unintegrated plasmid after this length of time.

The presence of genomically-inserted copies of the luciferase gene is tested in the hearts of DH2pGL3-UTMD-treated mice with PCR, using the tissue homogenates as template source and primers specific for the transgenic GL3 luciferase.

UTMD delivery of the luciferase transgene by the piggyBac insertional vector DH2pGL3 is repeated in another set of mice using these procedures, and the expression levels of luciferase in the heart is tested after 10, 50, and 100 days. Appearance of luciferase activity at these timepoints further verifies that the luciferase gene is maintained in the cells of the target tissue following piggyBac-mediated genomic insertion.

### Example 2

### Verification of Genomic Insertion of a PiggyBac Transgene Following Microbubble Delivery of the PiggyBac Insertional Plasmid

A version of the plasmid DH2pGL3 is prepared in which a kanamycin antibiotic resistance gene and bacterial origin of replication are located within the piggyBac transposon region, along with the GL3 gene, between the two terminal repeats utilized by the piggyBac transposase. Microbubble delivery of the plasmid to heart and liver tissue (in separate mice) is performed as described in Example 1. After 30 days, the mice are killed, their hearts or livers (as appropriate) removed, homogenized in buffer, and genomic DNA is recovered and assayed for gene insertion. This is achieved by digesting the genomic DNA with a restriction enzyme for which there is no cleavage site within the piggyBac transposon of DH2pGL3. The digested DNA is then religated and transformed into bacteria, which is then cultured in the presence of the antibiotic kanamycin. Bacteria that have been transformed with circularized genomic DNA containing a copy of the piggyBac transposon survive in these selective growth conditions by virtue of expression of the kanamycin resistance gene and the origin of replication on the transposon, which allows these circularized genomic sequences to behave as plasmids. This circularized DNA can be recovered like a plasmid, and primers specific for the transgene can be used to perform PCR walking from the transposon region into the genomic sequence into which the transposon originally inserted, thereby identifying the site of genomic integration.

### Example 3

Microbubble Delivery of a PiggyBac Insertional Plasmid to Liver tissue in Mice

Tissue-specific delivery of a luciferase reporter gene to the liver in mice is performed using the procedure of Example 1. Plasmid DH2pGL3 and the control plasmid SV40pGL3 are each combined with perfluorocarbon gas-filled cationic microbubbles as described in the previous example, and infusion of mice with the mixture is performed as described. In this case, ultrasound is directed at mice livers to disrupt the microbubbles and deliver the test plasmid to this location. After 2 or 28 days, the mice are killed, and their livers removed, homogenized and processed as described for hearts in Example 1 to assay for luciferase expression using the luciferase assay system (Promega). As observed for hearts, expression of luciferase in DH2pGL3-treated mice is observed after 2 days at levels significantly higher than observed in livers from mice treated with the SV40pGL3 control plasmid. Activity is also observed in liver tissue removed after 28 days from DH2pGL3-treated mice, while it is absent in livers from mice treated with the SV40pGL3 control plasmid.

### Example 4

### Microbubble Delivery of a PiggyBac Insertional Plasmid to Retinal Tissue in Mice

Tissue-specific delivery of a luciferase reporter gene to retinal cells in mice is performed using the procedure of Example 1. Plasmid DH2pGL3 and the control plasmid SV40pGL3 are each combined with perfluorocarbon gas-filled cationic microbubbles as described in the previous example, and infusion of mice with the mixture is performed as described. In this case, ultrasound is directed at retinal tissue in mice to disrupt the microbubbles and deliver the test plasmid to this location. After 2 or 28 days, the mice are anesthetized and injected with 150 mg per kg animal weight of luciferin (the substrate for firefly luciferase). Luciferase activity in the retina is then assayed by directly measuring luminescence in the eyes using an IVIS imaging system (Xenogen). Retinal expression of luciferase is higher after 2 days in mice treated with DH2pGL3 than in mice treated with the SV40pGL3 control plasmid. Activity is also observed in retinas 28 days following UTMD in DH2pGL3-treated mice, while it is absent in retinas from mice treated with the SV40pGL3 control plasmid.

### Example 5

Gene Therapy in Hemophiliac Mice using a Microbubble-Delivered PiggyBac Transgene encoding a Biotherapeutic Polypeptide (Factor VIII)

A piggyBac transposon-containing plasmid identical to DH2pGL3, but containing the gene for murine Factor VIII in place of GL3, is constructed. Hemophiliac mice (Factor VIII- knockout mice [Bi L, Lawler A M, Antonarakis S E, High K A, Gearhart J D, Kazazian H H, Jr. Targeted disruption of the mouse factor VIII gene produces a model of haemophilia A [letter]. Nat Genet. 1995, 10:119-121]) are given liver-targeted UTMD treatment as described in Example 1 using microbubbles loaded with this newly-constructed plasmid. Thirty days after treatment, plasma samples are obtained from each mouse by retro-orbital bleeding in 20% 0.1 M sodium citrate for functional murine Factor VIII determination (FVIII COA tests [Chromogenix, Molndal, Sweden]; see, Chuah M K, Vandendriessche T, Morgan R A. Development and analysis of retroviral vectors expressing human factor VIII as a potential gene therapy for hemophilia A. Hum Gene Ther. 1995, 6:1363-1377). Plasma from Factor VIII-knockout or Factor VIII-knockout SCID mice spiked with human plasma-derived Factor VIII (Octapharma, Langenfeld, Germany) of known activity was used as standard.

Supraphysiologic Factor VIII levels (>1000 mU/ml) are observed in the UTMD-treated mice. Control animals injected with empty microbubbules or microbubbles loaded with a version of pCMV-GL3 containing the murine Factor VIII gene in place of GL3 have no detectable Factor VIII (<25 mU/ml=background level).

To further assess phenotypic correction of the clotting deficiency, plasma samples are subjected to a functional clotting assay (aPTT) that measures the activated partial thromboplastin time (Hemoliance SythASil APTT Reagent, Lexington, Mass., USA). The inhibitory antibody titers are determined with Bethesda assays as described (Kasper C K, Aledort L, Aronson D, Counts R, Edson J R, van Eys J, Fratantoni J, Green D, Hampton J, Hilgartner M, Levine P, Lazerson J, McMillan C, Penner J, Shapiro S, Shulman N R. Proceedings: A more uniform measurement of factor VIII inhibitors. Thromb Diath Haemorrh. 1975, 34:612). Briefly, inhibition of Factor VIII activity by serially diluted mouse plasma is measured by using a functional Factor VIII COAtest. The detection limit of the assay is 0.2-0.4 Bethesda units/ml. for measuring cytokine and aminotransferase levels and for Bethesda assays., which are reduced by the treatment. The clotting time is significantly reduced in plasma obtained from hemophilic Factor VIII-kockout SCID mice that received UTMD treatment compared to the clotting time of plasma from untreated hemophilic Factor VIII-kockout-SCID control mice. Hence, gene therapy using microbubble delivery of a piggyBac insertional plasmid encoding funtional murine Factor VIII stably corrects the bleeding diathesis of these hemophilic mice.

### Example 6

Gene Therapy in Mice and Promoting Cardiac Angiogenesis using a Microbubble-delivered PiggyBac Transgene Encoding a Biotherapeutic Polypeptide (VEGF).

A piggyBac transposon-containing plasmid identical to DH2pGL3, but containing the gene for mouse vascular endothelial growth factor (VEGF) in place of GL3, is constructed. Ultrasound images of mouse cardiac vasculature are obtained, and then these mice are given heart-focused UTMD treatment as described in Example 1 using microbubbles loaded with this newly-constructed plasmid. Thirty days after treatment, cardiac vasculature is again imaged by ultrasound, and the extent of new cardiac blood vessel growth is observed, revealing noticeable new blood vessel formation. This is compared to new cardiac vasculature growth in mice treated with a non-insertional control plasmid (SV40pGL3 with the GL3 gene replaced by the gene for VEGF), which is not as extensive. This technique is then tested in atherosclerotic human patients to trigger new blood vessel formation around atherosclerotic plaque-containing cardiac arteries, thus bypassing blocked arteries.

### Example 7

Gene Therapy in Mouse Models of Cancer using a Microbubble-Delivered PiggyBac Transgene Encoding a Biotherapeutic p53 Polypeptide.

A piggyBac transposon-containing plasmid identical to DH2pGL3, but containing the gene for mouse p53 in place of GL3, is constructed. P53 knockout mice in which cancer has been triggered by exposure to a carcinogenic chemical are given UTMD treatment as described in Example 1, but focused at a particular organ or tissue with cancer growth, using microbubbles loaded with this newly-constructed plasmid. 50 days after treatment, the size and extent of tumor growth in the target tissue is examined, revealing it to be reduced following UTMD-mediated delivery of microbubbles loaded with the p53 gene-containing piggyBac plasmid. To test for genomic insertion of the p53 gene in these mice, the animals are killed, and the targeted tissue is removed and homogenized. Genomic DNA from the cancerous tissue is isolated from the homogenate, and the presence of the p53 gene is verified by PCR.

### Example 8

Gene Therapy in Mouse Models of Cancer using a Microbubble-Delivered PiggyBac Transgene Encoding a Biotherapeutic Ribonucleotide

A piggyBac transposon-containing plasmid identical to DH2pGL3, but containing a gene encoding a small inhibitory ribonucleic acid (siRNA) segment containing a short sequence matching a portion of the mRNA transcript for mouse vascular endothelial growth factor (VEGF) receptor, is constructed. P53 knockout mice in which cancer has been triggered by exposure to a carcinogenic chemical are given UTMD treatment as described in Example 1, but focused at a particular organ or tissue with cancer growth, using microbubbles loaded with this newly-constructed plasmid. 50 days after treatment, an ultrasound image of the vasculature within a tumor is obtained and compared to a similar image obtained prior to UTMD microbubble delivery of the plasmid. The size and extent of tumor growth in the target tissue is examined, revealing it to be reduced following UTMD-mediated delivery of microbubbles loaded with the VEGF receptor siRNA gene-containing piggyBac plasmid. To test for genomic insertion of the siRNA gene in these mice, the animals are killed, and the targeted tissue is removed and homogenized. Genomic DNA from the cancerous tissue is isolated from the homogenate, and the presence of the siRNA gene is verified by PCR.

### Example 9

Single-Plasmid PiggyBac-Mediated Transgenesis in Human HEK293 Cells using pMMK-1

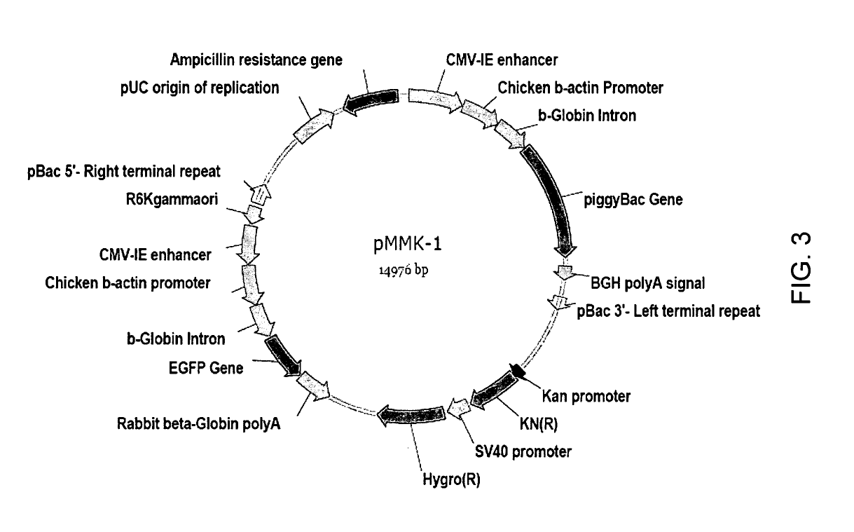

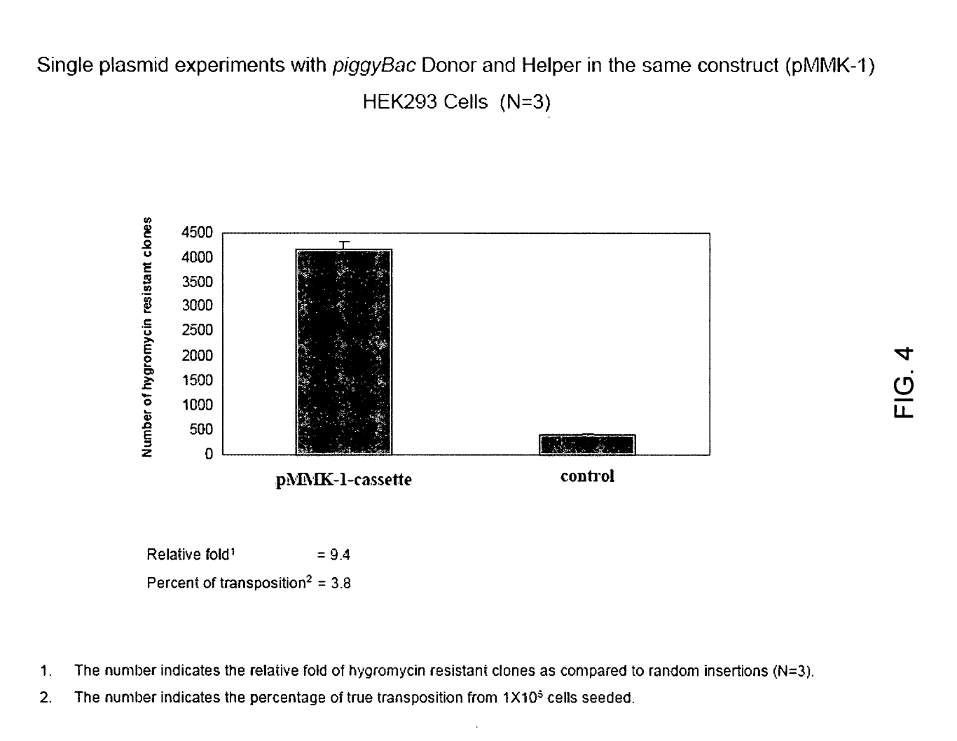

To demonstrate the efficiency of transgenesis in human cells using a single plasmid carrying both a piggyBac transposon and the gene for piggyBac transposase, HEK293 cells were transfected with the plasmid pMMK-1 (FIG. 3), which the hygromycin resistance gene on a piggyBac transposon and also encodes piggyBac transposase. HEK293 cells were maintained in MEM alpha medium (Hyclone) containing 5% FBS (Hyclone). Cells at 80% confluence were harvested, and 1×10⁵ cells were seeded into individual wells of 24-well plates 18 hours before transfection. A total of 400 ng of DNA were used for each transfection with FuGENE 6 (Roche). One-tenth of the transfected cells was transferred to 100 mm plates followed by hygromycin selection for 14 days. The concentration of hygromycin B used in HEK293 cells was 100 μg per milliliter. To count the number of clones, cell colonies were fixed with phosphate-buffered saline (PBS) containing 4% paraformaldehyde for 10 minutes and then stained with 0.2% methylene blue for 1 hr. After 14 days of hygromycin selection, only colonies larger than 0.5 mm in diameter were counted. FIG. 4 shows the number of hygromycin resistant colonies counted after transfection with pMMK-1 as compared with the number seen after transfection with a control plasmid lacking the piggyBac coding sequence. Transgenesis mediated by piggyBac encoded on pMMK-1 was 9.4-fold more frequent than random insertions in cells transfected with the control plasmid.

### Example 10

Generation of Transgenic Mice using a Single Plasmid Encoding PiggyBac Transposase and Carrying a PiggyBac Transposon Encoding an EGFP Reporter Protein

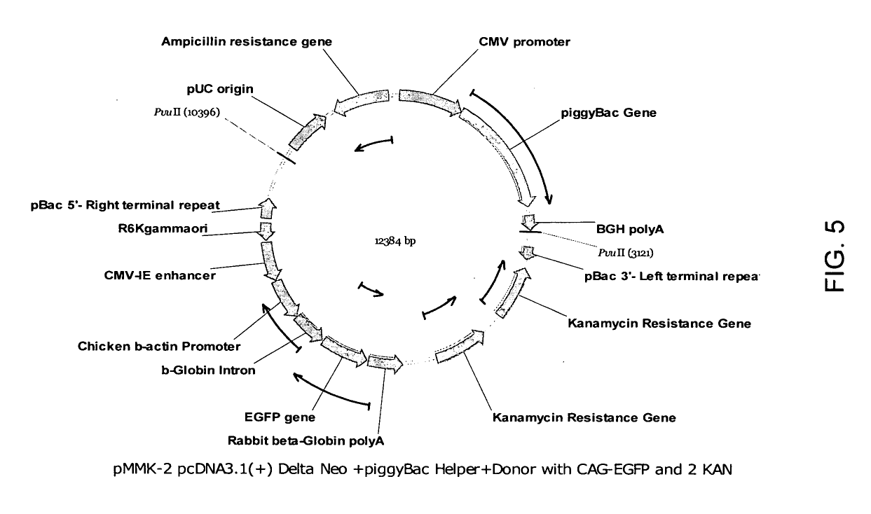

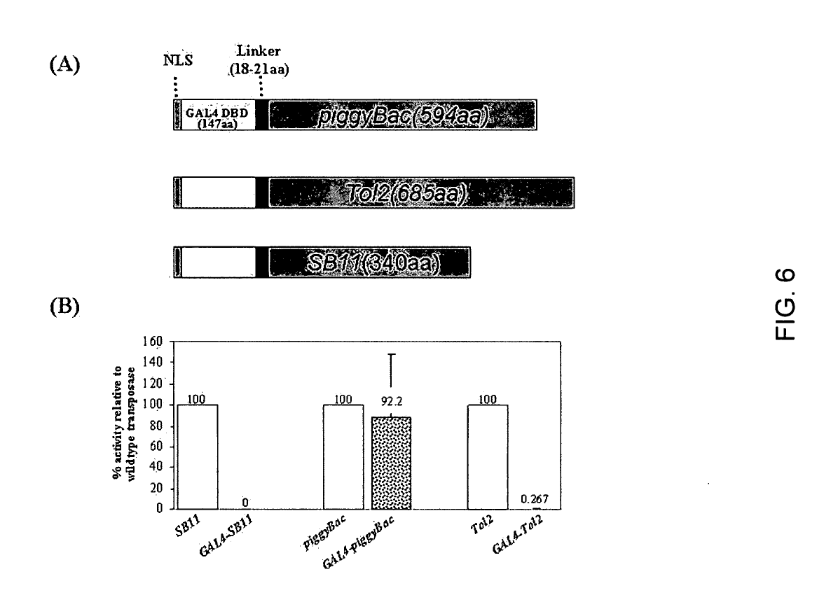

To demonstrate the efficiecy of piggyBac-mediated trangenesis in mouse embryos, EGFP-transgenic mice were generated using a compound plasmid encoding piggyBac transposase and carrying a piggyBac transposon encoding an EGFP reporter protein. Ten microliters of 200 nanogram/microliter plasmid pMMK-2 (FIG. 5), which contains the gene for EGFP on a piggyBac transposon and also encodes piggyBac transposase, was mixed with 10 microliters of fresh swim-up sperm solution. Each sperm head that had its tail removed in the mixed solution was individually microinjected into a metaphase II (MII) arrested matured mouse oocyte (intra-cytoplasmic sperm injection, ICSI). Alternatively, plasmid DNA alone was injected into a fertilized oocyte (pronuclear microinjection). Two-cell embryos were transferred into the oviducts of pseudopregnant females which were mated with vasectomized males the night before. The females were allowed to give birth to their own young. The newborn pups exhibit EGFP expression in their skin as observed by epifluorescence.

Table IV indicates the rates of transgenic mice generation (percentage of transgenic animals born for every oocyte injected) using the method of microinjection and concentration of plasmid pMMK-2 indicated.

TABLE IV
Transgenesis rates in mice after microinjection of plasmid pMMK-2 into mouse oocytes.

| Number of | | | Embryos | | | % Transgenic | |
|---|---|---|---|---|---|---|---|
| oocytes injected | Method | DNA concentration | transferred | Animals | Animals | (animals born) | |
| (repetitions) | (Promoter) | (volume injected) | (Surrogate #) | born | transgenic | [oocytes injected] | |
| 91 (1) | Pronuclear | 1 ng/μL (15 μm) | 61 (2) | 11 | 0 | — | — |
| | (CAG) | | | | | | |
| 159 (1) | Pronuclear | 10 ng/μL (15 μm) | 122 (3) | 15 | 4^{C} | (26.6) | [2.5] |
| | (CAG) | | | | | | |
| 74 (2) | ICSI (CMV) | 100 ng/μL (15 μm) | 63 (5) | 38 | 1^{C} | (2.6) | [1.4] |
| 104 (3) | ICSI (CAG) | 100 ng/μL (15 μm) | 82 (5) | 49 | 6^{C} | (12.2) | [5.8] |
| 36 (1) | ICSI (CAG) | 100 ng/μL (50 μm) | 27 (2) | 13 | 4^{C} | (31.0) | [11.1] |
| 33 (2) | ICSI (CAG) | 100 ng/μL (100 μm) | 29 (2) | 9 | 4^{C} | (44.4) | [12.1] |
| 79 (2) | ICSI (CAG) | 150 ng/μL (100 μm) | 69 (4) | 26 | 18^{C} | (69.2) | [22.8] |
| 72 (2) | ICSI (Donor | 150 ng/μL (100 μm) | 57 (3) | 19 | 0 | 0 | 0 |
| | only) | | | | | | |

### Example 11

Activity of a GAL4 PiggyBac Chimeric Transposase is Similar to that of the Wild Type Transposase

Directing transgene integration to a unique and safe site on the host chromosome can overcome the hazards of insertional mutagenesis that can result with integrating vectors currently in use. A transposon-based gene delivery system preferably features a custom-engineered transposase with high integration activity and target specificity. Targeting transposon integration to specific DNA sites using chimeric transposases engineered with a DNA binding domain (DBD) has been demonstrated in mosquito embryos containing a plasmid including a unique site recognized by a GAL4 DNA binding domain fused to a transposase (Maragathavally, K. J. et al, FASEB J. [2006] 20:1880, which is hereby reference by incorporation in its entirety). Such modifications can render a transposase inactive, however, as observed for variants of SB11 engineered for target specificity, which have dramatically reduced transposition activity (Wilson, M. H. et al, FEBS Lett [2005] 579: 6205). Therefore, the potential for modifications of SB11, Tol2, and piggyBac transposases was assessed by testing their activity when fused to a GAL4 DNA binding domain. The transposition activities of each of these transposases, upon addition of an N-terminal GAL4 DBD (FIG. 12A), was determined using a chromosome integration assay in HEK293 cells. GAL4 piggyBac transposase demonstrated transposition activity similar to that of wild-type piggyBac, while GAL4-Tol2 and GAL4-SB11 transposases possessed negligible activity (FIG. 12B), even though GAL4-SB11 protein was detected by Western blot using a monoclonal antibody.

PiggyBac inserts into the tetranucleotide site TTAA, which is duplicated upon insertion (Ding, S. et al, Cell [2005] 122: 473; Tosi, L. R. et al, Nucleic Acids Res. [28: 784). To test whether fusion of GAL4 to the N-terminus of piggyBac transposase alters its preference for TTAA sites, plasmid rescue experiments were performed to retrieve the sequence information of the target sites using genomic DNAs isolated from individual hygromycin-resistant CHO cell clones. Individual clones were isolated and allowed to grow to confluence in a 100mm plate. Genomic DNA was isolated using a DNeasy Tissue kit according to the manufacturer's protocol (Qiagen). Five micrograms of genomic DNA was subjected to Xhol digestion followed by ligation into a plasmid containing a bacterial origin of replication and an antibiotic resistance gene. The ligation reactions were transformed into E. coli DH10B cells. Plasmids rescued from transformants were subjected to DNA sequencing to retrieve the genomic sequence flanking the insertion site. Six independent genomic sequences were recovered from four drug-resistant clones. As shown in Table V, all of these sequences contain genomic DNA with the signature TTAA sequence at the integration site. This experiment demonstrates that the chromosomal integrations observed in cells transfected with GAL4 piggyBac are mediated by a true transposition event with the same insertion preference for TTAA sites. Thus, neither the mechanism of transposon insertion by piggyBac transposase, nor its high level of activity appear, appear to be effected by fusion to a site-selective GAL4 DBD.

TABLE V
Analysis of chromosomal insertion
site selection by GAL4-piggyBac
transposase in CHO cells

| | Donor | | |
|---|---|---|---|
| Independent | plasmid | Chromosomal | Flanking |
| Isolated | terminal | Insertion | Chromosomal |
| Clones | repeat | Site | Sequence |
| G8-2 | ^{5′}TGATTATC | TTAA | GCTCGGGCCGGCCG |
| | TTTCTAGGG | | CGTCGCCGCTTC^{3′} |
| G25-2 | TGATTATCT | TTAA | CAATCAATAAGAT |
| | TTCTAGGG | | AAACATACACAGA |
| G25-3 | TGATTATCT | TTAA | CACCACATTTAAC |
| | TTCTAGGG | | TTGCTCTTTGATA |
| G28-1 | TGATTATCT | TTAA | TAGAGTGCTGAGA |
| | TTCTAGGG | | TTTGGGACATTGC |
| G29-1 | TGATTATCT | TTAA | GGCGTTGGTGGCA |
| | TTCTAGGG | | CACAACTTTAAGT |
| G34-2 | TGATTATCT | TTAA | TAAGACAATGTAT |
| | TTCTAGGG | | GACTTTGTCCCAT |

### Example 12

PiggyBac Exhibits Greater Transposition Activity in Mammalian Cells than SB11, Tol2, and Mos1

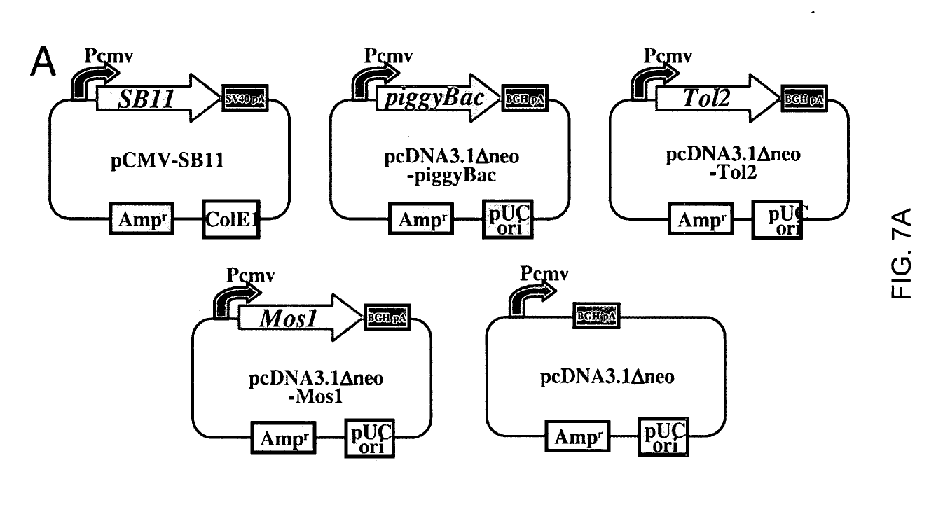

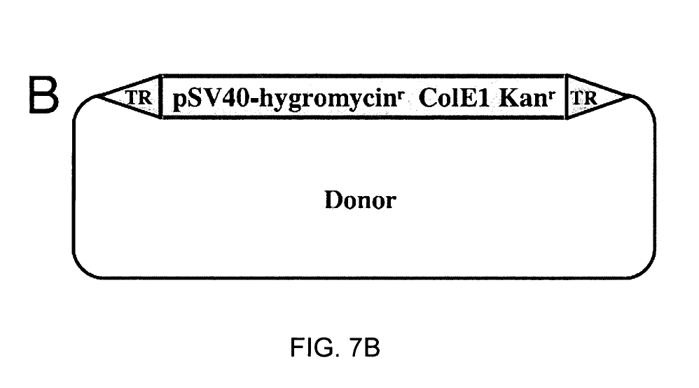

Since different transposon systems have been independently developed and tested in different laboratories, so a direct comparison of transposition activity of various transposon systems identified the most promising transposon(s). Mos1, SB11, Tol2, and piggyBac transposon systems were be constructed using a two-component system (FIG. 7): a helper plasmid containing the transposase driven by the cytomegalovirus (CMV) promoter (FIG. 7A) and a donor plasmid with the terminal repeats bearing a cassette with hygromycin resistance and kanamycin resistance genes to facilitate selection in eukaryotes and prokaryotes, respectively, and a ColE1 replication origin for plasmid propagation in bacteria (FIG. 7B). Transposition activity of each of the four transposon systems was determined in four mammalian cell lines: HeLa (human cervical carcinoma), HEK293 (human embryonic kidney cell), H 1299 (human lung carcinoma), and CHO (Chinese hamster ovarian carcinoma).

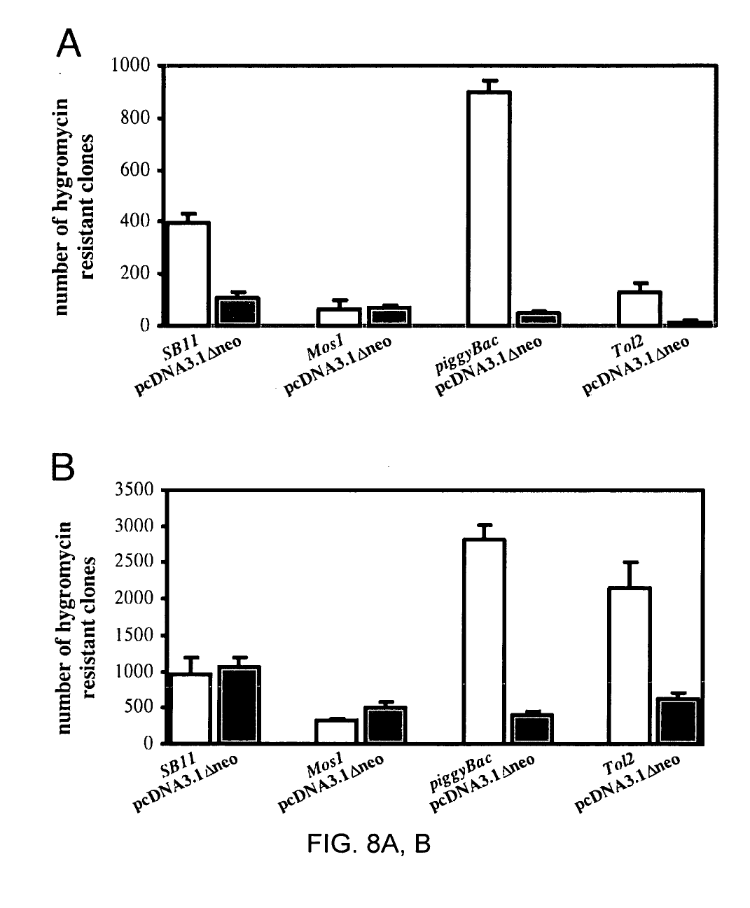

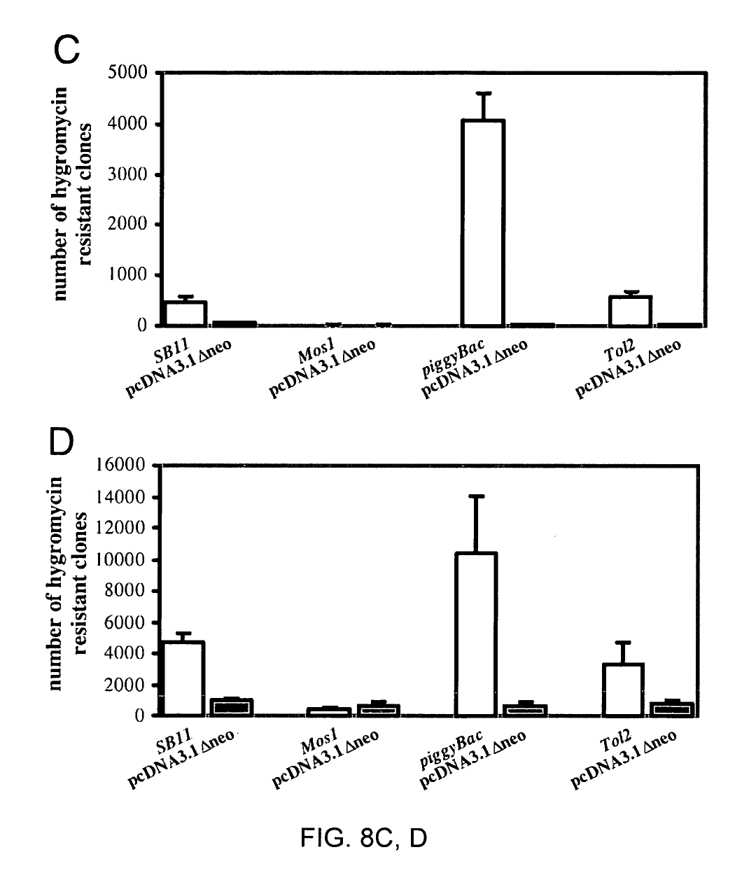

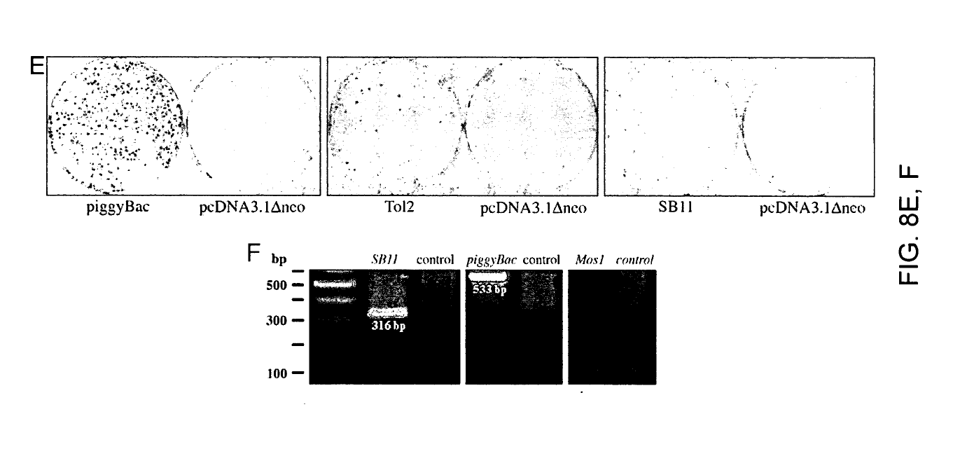

To assess efficiency of transgenesis, cells at 80% confluence were harvested, and 1×10⁵ cells were seeded into individual wells of 24-well plates 18 hours before transfection. A total of 400 ng of DNA was used for each transfection reaction with FuGENE 6 (Roche). For each cell line, one-tenth of the transfected cells was transferred to 100 mm plates followed by hygromycin selection for 14 days. The concentration of hygromycin B used in HeLa, HEK293, H1299, and CHO cells was 200, 100, 400, and 400 μg per milliliter, respectively. To count the clones, cells were fixed with PBS containing 4% paraformaldehyde for 10 min and then stained with 0.2% methylene blue for 1 hr. After 14 days of hygromycin selection, cell colonies were counted. Because colonies smaller than 0.5 mm in diameter often fail to be subcloned in the presence of hygromycin, only colonies larger than 0.5 mm in diameter were counted. As shown in FIG. 8 (A-E), piggyBac and Tol2 possessed activity in all cell lines tested. SB11 displayed slight transposition activity in CHO, HeLa, and HEK293 cells, while it is inactive in H1299 cells. No transposition activity was detected with Mos] in the four cell lines used (FIG. 8A-E).

As indicated in FIG. 8A-D, the transposition activity of piggyBac, Tol2, and SB11, varied in different cells. For example, ˜1000 hygromycin-resistant colonies were detected with both the control plasmids and SB11-expressing plasmid in H 1299 cells (FIG. 8B), suggesting a lack of transposition activity of SB11 in this cell line. However, in HEK293 cells, ˜500 hygromycin-resistant colonies were detected in the presence of SB11 transposase, which represents an 8-fold increase over cells transfected with control plasmid (FIG. 8C). Two parameters, relative fold and percentage of transposition, were thus used to assess the transposition activity of the different transposon systems. The relative fold was obtained by dividing the number of hygromycin-resistant colonies detected in cells transfected by donor plus helper by the colony number that results from random integration (as in transfection experiments with control plasmid not encoding a transposase). The percentage of transposition, hereafter referred to as transposition rate, was calculated by subtracting the number of hygromycin-resistant colonies detected in cells transfected with control plasmid from the number of resistant colonies observed in cell transfected with a transposase-encoding plasmid, then dividing by 1×10⁵ (the number of cells originally seeded before transfection), and finally multiplying by 100 to convert to percentage. The transposition rate represented here, however, was not normalized by the transfection efficiency in various cell lines. As summarized in Table VI, the relative fold ranges for the three transposons in different cell lines were as follows: (1) SB11 from 1 [equal to control] in H1299 to 8.1 in HEK293, (2) piggyBac from 5.7 in H1299 to 114 in HEK293, and (3) Tol2 from 3.3 in CHO to 93.9 in HEK293. The transposition rate ranges were: (1) SB11 from 0% in H1299 to 2.9% in CHO, (2) piggyBac from 0.7% in HeLa to 7.0% in CHO, and (3) Tol2 from 0.08% in HeLa to 1.8% in CHO. Once again, piggyBac displayed the highest transposition activity among the three active transposon systems tested, as judged by both the transposition rate and relative fold. The transposition rate of Tol2 was higher than SB11 in H1299 and HEK293 but not in CHO and HeLa cells. Owing to the relatively high integration rate of the SB11 control, the relative fold seen in all four cell lines for Tol2 was higher than that of SB11.

TABLE VI
Summary of the transposition efficiency of SB11, piggyBac, and Tol2 transposon systems.

| | SB11 | | piggyBac | | Tol2 | |
|---|---|---|---|---|---|---|
| | Relative | Percentage of | Relative | Percentage of | Relative | Percentage of |
| Cells | fold | transposition | fold | transposition | fold | transposition |
| CHO | 2.9 ± 1.6 | 2.3 ± 1.5 | 9.8 ± 6.4 | 7.0 ± 3.9 | 3.3 ± 1.7 | 1.8 ± 1.2 |
| HeLa | 2.7 ± 1.0 | 0.2 ± 0.1 | 14.5 ± 3.5 | 0.7 ± 0.2 | 5.4 ± 3.2 | 0.08 ± 0.03 |
| H1299 | 1.0 ± 0.16 | 0 | 5.7 ± 1.3 | 2.2 ± 0.2 | 4.1 ± 0.8 | 1.4 ± 0.3 |
| HEK293 | 8.1 ± 2.1 | 0.4 ± 0.1 | 114.6 ± 81.6 | 2.8 ± 1.4 | 93.9 ± 75.8 | 0.9 ± 0.3 |

Relative fold values indicate the relative fold of hygromycin-resistant clones as compared with controls (n = 6).
Percentage of transposition values indicate the percentage of true transposition from 1 × 105 cells seeded.

The type of DNA transposition described herein involves a two-step action: (1) excision of the transposable element from the donor plasmid, and (2) integration of the excised fragment into its DNA target. Therefore, the numbers of hygromycin-resistant colonies are the result of both excision and integration events. Although no activity was detected in cells transfected with Mos1, it was still possible that successful excision occured but that integration did not. To exclude this possibility, a plasmid-based excision assay was performed using the polymerase chain reaction (PCR). As a consequence of excision, a short version of the donor plasmid should be produced.

To perform this assay, 1×10⁶ HEK293 cells were seeded onto 60 mm² plates 18 hours before transfection. One microgram each of donor and helper plasmid was added to the media to transfect the cells. Plasmids were recovered using the Hirt method 72 hours after transfection (Ziegler, K. et al. J. Virol. Methods [2004] 122: 123). Plasmids isolated were used as templates for nested PCR using primers listed below to detect the presence of donor plasmids that undergo excision: piggyBac first round, 5Bac-1(TCGCCATTCAGGCTGCGC)/3Bac-1(TGTTCGGGTTGCTGATGC); piggyBac second round, 5Bac-2(CCTCTTCGCTATTACGCC)/3Bac-2 (TGACCATCCGGAACTGTG); Sleeping Beauty first round, F1-ex (CCAAACTGGAACAACACTCAACCCTATCTC)/o-lac-R(GTCAGTGAGCGAGGAAGCGGAAGAG); Sleeping Beauty second round, KJCO31(CGATTAAGTTGGGTAACGCCAGGGTTT)/i-lac-R(AGCTCACTCATTAGGCACCCCAGGC); Mos1 first round, 5mos-1 (TCCATTGCGCATCGTTGC)/3mos-1 (AGTACTAGTTCGAACGCG); Mos/second round, 5mos-2 (ACAGCGTTGTTCCACTGG)/3mos-2 (AAGCTGCATCAGCTTCAG).

No excision-dependent PCR product was detected in cells transfected with donor and helper plasmids for Mos1, whereas excision-dependent PCR products with sizes of 533 by for SB11 or 316 by for piggyBac were detected (FIG. 8F). SB11 and piggyBac are therefore able to both excise and integrate their respective transposons, while Mos/is unable to do either.

1. A method for tissue-specific delivery of a gene therapeutic, transgenic nucleic acid in a mammal, comprising the steps of:
providing a nucleic acid comprising a transgene flanked by two terminal repeats and, within the same or on a separate nucleic acid, a nucleotide sequence encoding a transposase, wherein the transgene comprises a biotherapeutic gene;
contacting said nucleic acid with perfluorocarbon gas-filled microbubbles to form a mixture;
introducing said mixture into the bloodstream of a mammal; and
focusing ultrasound pulses on a specific tissue of said mammal, wherein said pulses disrupt said microbubbles of said mixture and release said nucleic acid into the bloodstream within the target tissue, thereby enabling uptake of the transgenic nucleic acid into the cells of said target tissue.

2. The method of claim 1, wherein said transgene encodes a biotherapeutic polypeptide.

3. The method of claim 1, wherein said .transgene encodes a biotherapeutic ribonucleic acid product.

4. The method of claim 2, wherein said transposase can be selected from one of the group consisting of piggyBac, Sleeping Beauty, Mos1, Tc1/mariner, Tol2, Tc3, MuA, and Himar1 transposase.

5. The method of claim 2, wherein said nucleic acid comprising a transgene flanked by two terminal repeats is a piggyBac-like transposon and said transposase is apiggyBac-like transposase.

6. The method of claim 1, wherein said transgene is under the control of a promoter.

7. The method of claim 1, wherein said transgene is under the control of one of the group consisting of the CMV promoter and CAG promoter.

8. The method of any of claim 5, wherein said piggyBac-like transposase is a chimeric transposase comprising a host-specific DNA binding domain.

9. The method of claim 8, wherein the host-specific DNA binding domain of said chimeric transposase comprises a Saccharomyces cerevisiae Gal4 zinc finger DNA-binding protein.

10. The method of either claim 8, wherein the host-specific DNA binding domain of said chimeric transposase is optimized for host specificity.

11. The method of any claim 1, wherein the transgene comprises a selectable marker or reporter gene.

12. The method of claim 11, wherein the selectable marker or reporter gene is selected from the group consisting of EGFP, luciferase, and β-galactosidase.

13. The method of claim 8, wherein the host-specific DNA binding domain of the chimeric transposase is fused to the N-terminus of said transposase.

14. The method of claim 8, wherein the host-specific DNA binding domain of the chimeric transposase is fused to the C-terminus of said transposase.

15. The method of claim 1, wherein said mammal is selected from one of the group consisting of primates, rodents, cows, pigs, sheep, goats, horses.

16. The method of claim 1, wherein said mammal is a human.
